(19) United States
(12) Patent Application Publication
(10) Pub. No.: US 2018/0346525 A1
(43) Pub. Date: Dec. 6, 2018

(54) DIAGNOSTIC AND THERAPEUTIC QUORUM-SENSING-MANIPULATION MOLECULES THAT ARE TRACKABLE FOR HEALTH-CARE AND INDUSTRIAL SYSTEMS.

(71) Applicant: The Trustees of Princeton University, Princeton, NJ (US)

(72) Inventors: Bonnie L. Bassler, Princeton, NJ (US); Howard A. Stone, Princeton, NJ (US); Min Young Kim, Seoul (KR); Aishan Zhao, Princeton, NJ (US); Thomas William Muir, Princeton, NJ (US)

(73) Assignee: The Trustees of Princeton University, Princeton, NJ (US)

(21) Appl. No.: 15/950,217

(22) Filed: Apr. 11, 2018

Related U.S. Application Data
(60) Provisional application No. 62/484,439, filed on Apr. 12, 2017.

(51) Int. Cl. C07K 14/21; C07K 16/28; C12Q 1/02

(52) U.S. Cl. CPC C07K 14/21; C07K 16/28; C12Q 1/025; A61L 2300/606; G01N 2333/21; A61L 2420/02; A61L 2300/404

(57) ABSTRACT

According to present invention embodiments, a trackable moiety can be attached to a quorum sensing (QS) molecule to form a QS modulating conjugate. QS modulating conjugates retain their activity for QS manipulation and are able to be detected by imaging techniques. The QS portion of the QS modulating conjugate can play a role in affecting bacterial behaviors, such as, inhibition of biofilms or disruption of toxin production, while the trackable moiety of the QS modulating conjugate enables monitoring, visualization in real time of its binding to the receptor on the bacterial surface, and the location of the bacterium itself, for example, in a biofilm and/or at an infection site. Since binding of the QS modulating conjugate to its cognate receptor is specific, the QS modulating conjugate can be used for diagnostic applications by enabling pinpointing of specific bacteria at infection sites.

### CROSS-REFERENCE TO RELATED APPLICATIONS

This application is claims priority to U.S. 62/484,439 filed on Apr. 12, 2017, which is hereby incorporated by reference in its entirety.

### STATEMENT REGARDING FEDERALLY SPONSORED RESEARCH OR DEVELOPMENT

This invention was made with government support under Grant No MCB-1344191 and MCB-0948112 awarded by the National Science Foundation and Grant No. GM-065859 awarded by the National Institutes of Health. The government has certain rights in the invention.

### BACKGROUND

In a process referred to as quorum sensing (“QS”), microorganisms, such as bacteria, communicate using extracellular chemical signaling molecules called autoinducers. QS involves the production, release, and population-wide detection of autoinducers¹⁻⁸. By monitoring increases and decreases in autoinducer concentration, QS bacteria track changes in cell-population density and synchronously switch into and out of collective group behaviors. QS also allows bacteria to collectively carry out tasks that would be unsuccessful if carried out by an individual bacterium acting alone.

Both Gram-positive and Gram-negative infectious bacteria, which include human, animal, plant, and marine pathogens, use QS to control virulence. QS also controls biofilm formation and in some cases streamer formation. Biofilms are communities of bacterial cells adhered to surfaces and encased in a self-produced matrix of extracellular polymeric substances. In most environments, bacteria are found predominantly in biofilms. These biofilms are also widespread in industrial systems and are associated with increased risk of infection when found in clinical environments and in indwelling medical devices. These bacterial biofilm communities can cause chronic infections in humans by colonizing, for example, in medical implants, heart valves, or lungs.

In settings involving fluid flow across the biofilm, as in the environment, for example, in rivers or in industrial and medical systems that are subject to flow, filamentous biofilms, called streamers, can be formed. These streamers can have a dramatic effect on the biofilm environment. In rivers, for example, the biofilm streamers can increase transient storage and cycling of nutrients and can enhance the retention of suspended particles. In industrial and medical settings, the biofilm streamers have been associated with increased issues associated with clogging and pressure drops.

Bacterial infections are typically treated with bactericidal or bacteriostatic molecules that impede one or more of at least five major processes: cell wall formation, DNA replication, transcription, translation or tetrahydrofolic acid synthesis. Existing methods for treating bacterial infection unfortunately exacerbate the growing antibiotic resistance problem because they inherently select for growth of bacteria that can resist the drug.

Staphylococcus aureus is a human pathogen notorious for causing hospital-acquired infections, most of which are fatal. S. aureus infections are of primary concern because S. aureus forms biofilms, produces virulent toxins, and is responsible for multiple fatal diseases including bacteremia, toxic shock syndrome, and medical device-related infections. Many strains of S. aureus are multi-drug resistant (i.e. Methicillin-resistant Staphylococcus aureus (MRSA))^{24,25}. In this context, the quorum-sensing system plays a central regulatory role in S. aureus pathogenicity and biofilm dynamics^{1,8}. Specifically, pro-QS molecules activate toxin production and promote biofilm dispersal in S. aureus, while anti-QS molecules perform the reverse.

Another problematic species is Pseudomonas aeruginosa, a pathogen that can survive in a wide range of environments. The bacterium is a public health threat because it causes a variety of secondary infections in humans, where those with burn wounds, cystic fibrosis, and implanted medical devices and cancer patients receiving chemotherapy are particularly at risk. With an outer membrane of low permeability, a multitude of efflux pumps, and various degradative enzymes to disable antibiotics, P. aeruginosa is difficult to treat, and rapid diagnostic tests are not currently available to detect this pathogen. As with other common pathogenic bacteria, antibiotic-resistant strains are an increasing problem.

S. aureus infections that are associated with abiotic materials, such as intravenous catheters and implants, are of primary concern as S. aureus readily colonizes such medical devices, forming biofilms, biofilm streamers, and initiates virulence factor production under these conditions. S. aureus is just one example of a microorganism that uses quorum-sensing-mediated communication to control virulence factor production and to regulate biofilm formation.

Determining whether a patient has a specific type of infection caused by a particular microorganism is often a time consuming and slow process, and in some cases, taking several days for cell culture results to be ready. Additionally, it is frequently not clear where the source of the infection is in vivo.

Thus, what is needed are methods and compositions to detect particular types of microorganisms, to establish a location (e.g., within the human body) of the particular type of microorganisms, which can be effectively delivered without causing adverse side effects. Preferably, these methods will demonstrate improved efficacy and safety over conventional methods.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Presented herein are novel methods and molecules that exploit QS control of toxin production to inhibit the pathogenic behaviors of microorganisms, such as S. aureus, at infectious sites in patients. This method comprises conjugating pro- and anti-QS molecules to trackable moieties such as fluorescent molecules, radionuclides and PET probes. In some embodiments, a fluorophore, PET probe, or other type of trackable moiety can be attached to the pro- or anti-QS molecule to form a QS modulating conjugate. The QS modulating conjugate with QS antagonist activity could alter QS, for example, by ultimately down-regulating S. aureus toxin synthesis to generate a type of anti-staphylococcal medicine. Because the QS modulating conjugate also possesses a fluorescent probe, radionuclear probe, PET probe, or other detectable component, the QS modulating conjugate can be tracked. Thus, points of infection or any location containing a bacterium, such as for example, S. aureus, can be visualized in real time.

According to present invention embodiments, a trackable moiety can be attached to a QS molecule to form a QS modulating conjugate. QS modulating conjugates retain their activity for QS manipulation and possess the capability that allows tracking while holding biological and therapeutic promise. First, the QS portion of the QS modulating conjugate can play a role in affecting bacterial behaviors, such as, inhibition of biofilms or disruption of toxin production. Second, the tracking portion of the QS modulating conjugate enables it to be monitored, visualizing in real time its binding to the receptor on the bacterial surface, as well as the location of the bacterium itself, for example, in a biofilm and/or at an infection site. Third, because binding of the QS portion of the QS modulating conjugate to its cognate receptor is specific, the hybrid molecule can be used for diagnostic applications by enabling pinpointing of specific types bacteria at infection sites. For example, targeting of the infection site and identification of the particular bacterium S. aureus could provide appropriate information for initial and follow-up treatment, since S. aureus and MRSA cause a variety of fatal infections ranging from minor skin infections to serious illnesses such as infections of indwelling medical devices, osteomyelitis, endocarditis, sepsis, and toxic shock syndrome. Therefore, engineering dual function QS modulating conjugates can provide both therapeutic and diagnostic methods to potentially treat infectious diseases. Finally, QS modulating conjugates that bind and are trackable but that do not alter bacterial behavior are also useful for diagnostic/identification of microorganisms, especially pathogenic microorganisms.

Thus, present invention embodiments include a process that comprises combining imaging or other visualization technologies with a QS modulating conjugate comprising a QS modulator molecule involved in bacterial cell-to-cell communication in order to inhibit mechanisms that contribute to sepsis and other bacterial virulence functions as well as allow the infectious site(s) to be pinpointed. In some embodiments, the S. aureus Agr QS modulating conjugate is highly selective, binding only to S. aureus cells, enabling S. aureus to be selectively identified, e.g., S. aureus can be selectively identified in the presence of other species of bacteria so that only S. aureus is detected, while other species of bacteria are not. This feature has ramifications for rapid identification/diagnostics in the context of infection and/or in industrial applications.

It is expressly understood that the present invention embodiments are not limited to S. aureus, but may include any other QS bacterial species so long as the QS modulating molecule is known and binds specifically to a target receptor, and is amenable to chemistry, e.g., attachment to a trackable moiety.

Preferred examples of altered QS phenotypes (also referred to as traits) include, but are not limited to, significant reductions in biofilm formation, biofilm streamer formation, and virulence factor production. This technology can be immediately applied to many current and urgent issues in healthcare settings, such as detection of bacterial infections. This technology can also be used to determine whether a treatment of a bacterial infection is working, and if the treatment is considered not to be working alternate therapies (e.g., a different antibiotic) can be prescribed. Beyond medicine, this technology can also be applied to other fields including, but not limited to, industrial and engineering processes, detection of bacterial organisms in food processing, and other industrial settings.

Thus, present invention embodiments relate to a method of (1) conjugating a trackable moiety to an antagonist or antagonist of QS to form a QS modulating conjugate, (2) contacting the QS modulating conjugate with a biological sample comprising one or more microorganisms, and (3) detecting the microorganism that specifically binds to the QS modulating conjugate. A microorganism that is exposed to the antagonist or agonist exhibits altered biofilm production, biofilm streamer production, and/or virulence factor production. In some embodiments, conjugating anti-QS molecules to trackable moieties will lead to a detectable decrease in biofilm production, biofilm streamer production, and/or virulence factor production. In other embodiments, conjugating pro-QS molecules to trackable moieties will lead to a detectable decrease in biofilm production, biofilm streamer production, and/or virulence factor production. The QS modulating conjugate can be used for diagnostic applications as it specifically attaches to Agr quorum-sensing receptors on S. aureus (for example), and not on other types of microorganisms such as, for example, P. aeruginosa or V. cholerae. In the case of cystic fibrosis, the major microorganisms that are found in the lung are S. aureus, P. aeruginosa, or both. In the case of bacterial gastroenteritis, the major microorganisms that are found in the intestine are V. cholerae, S. aureus, or both. Thus, in these examples, S. aureus can be distinguished from other bacterial species in mixtures of bacterial species. Furthermore, the QS modulating conjugate may have additional therapeutic functions, since the autoinducer portion of the hybrid molecule can inhibit toxin production and/or disperse biofilms and/or change any other QS-controlled phenotypes.

For example, a QS modulating conjugate can be used to promote or inhibit pathogenic behavior of a microorganism in a patient as well as detect the type of microorganism responsible for the infection. By conjugating a QS modulating molecule to a trackable moiety to form a QS modulating conjugate, the QS modulating conjugate can promote or inhibit QS, in turn, leading to an alteration in biofilm formation, biofilm streamer formation, and/or virulence factor production.

Additionally, a QS modulating conjugate can be used to promote beneficial behaviors of the microorganism in a variety of settings, including, but not limited to, in food processing, engineering or industrial settings. The QS modulating conjugate, which controls QS regulated beneficial phenotypes including, but not limited to, enzyme or metabolite production, such as enzymes that can degrade plastics and petroleum products, enzymes that help digestion in humans, and metabolites that can be consumed by animals or humans, can be detected in a variety of environments.

In specific microorganisms, a QS agonist conjugate can repress biofilm formation and/or virulence factor expression. These microorganisms are virulent at low cell density and in response to QS autoinducers, can escape the host cell defenses. For example, Vibrio cholerae dissociates from the host's epithelial cells at high cell densities to become extremely contagious. In this situation, a QS agonist conjugate, rather than a QS antagonist conjugate, could be used to inhibit biofilm formation and thus repress virulence. Examples of such microorganisms include, but are not limited to S. aureus, Vibrio cholerae, Vibrio parahaemolyticus, Vibrio vulnificus, and Vibrio harveyi.

In other specific microorganisms, a QS antagonist conjugate can repress biofilm formation and/or virulence factor expression. These microorganisms are virulent at high cell density, and in response to QS autoinducers, can damage the host cells. In this situation, a QS antagonist conjugate, rather than a QS agonist conjugate, could be used to inhibit biofilm formation and/or repress virulence. Examples of such microorganisms include, but are not limited to Pseudomonas aeruginosa and Enterococcus faecalis.

As used herein, a biofilm, a biofilm streamer, and/or a virulence factor are produced or formed by a microorganism(s). In preferred embodiments, the microorganism is selected from the following groups: bacteria, archaea, protozoa, fungi, and/or algae. In further embodiments, the bacteria, archaea, protozoa, fungi, and/or algae are pathogenic to humans, animals and/or plants. Alternatively, the bacteria, archaea, protozoa, fungi, and/or algae are beneficial to humans, animals and/or plants. In further embodiments the bacteria, archaea, protozoa, fungi, or algae are common to industrial settings, including, but not limited to, industrial fluid handling processes, medical processes, agricultural processes, and/or machinery. In further embodiments, the bacteria, archaea, protozoa, fungi, or algae are common to an apparatus and/or process that involve fluid flow.

In still further embodiments, the bacteria are selected from the following genera: Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, and Yokenella.

In still further embodiments the bacteria are selected from the following species: Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, and Zymomonas mobilis.

In still further embodiments, the bacteria are from the class of bacteria known as Fusospirochetes. In further embodiments, the microorganism comprises fungi. In still further embodiments, the fungi are selected from the following genera: Candida, Saccharomyces, and Cryptococcus.

Such pathogenic bacteria can cause bacterial infections and disorders related to such infections that include, but are not limited to, the following: acne, rosacea, skin infection, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Peptostreptococcus spp. or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like. The QS modulating conjugates as described herein can be used to treat any of these disorders.

In certain embodiments the disease or disorder that can be treated with QS modulating conjugates as described herein include sepsis, pneumonia, lung infections from cystic fibrosis, otitis media, chronic obstructive pulmonary disease, a urinary tract infection, and/or combinations thereof. In other embodiments, the QS modulating conjugates described herein can be used to reduce and/or eliminate a medical device-related infection. In further embodiments, the QS modulating conjugates described herein can be used to treat a periodontal disease, such as gingivitis, periodontitis or breath malodor. In still further embodiments, the QS modulating conjugates described herein can be used to treat infections, including but not limited to those infections caused by bacteria. In some embodiments, the bacteria are Gram-negative or Gram-positive bacteria. Non-limiting examples of diseases and/or disorders that can be treated and/or prevented with the QS modulating conjugates include otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and cystic fibrosis.

In one specific example, subjects with cystic fibrosis can display with an accumulation of biofilm in the lungs and digestive tract. Subjects afflicted with COPD, such as emphysema and chronic bronchitis, display a characteristic inflammation of the airways wherein airflow through such airways, and subsequently out of the lungs, is chronically obstructed. Infections, including biofilm-related disorders, also encompass infections on implanted/inserted devices, medical device-related infections, such as infections from biliary stents, orthopedic implant infections, and catheter-related infections (e.g., kidney, vascular, peritoneal, etc.). An infection can also originate from sites where the integrity of the skin and/or soft tissue has been compromised. Non-limiting examples include dermatitis, ulcers from peripheral vascular disease, burn injury, and trauma. All of these diseases and/or disorders can be treated using the QS modulating conjugates as described herein.

A QS modulating conjugate (e.g., an antagonist) as described herein can be used to inhibit QS, thereby inhibiting biofilm formation, biofilm streamer formation and/or virulence factor expression in the healthcare field, in waste water treatment facilities or to treat those microorganisms that up-regulate these traits in response to QS autoinducers. A QS modulating conjugate (e.g., an agonist) as described herein can be used to promote QS thereby inhibiting biofilm formation, biofilm streamer formation and/or virulence factor expression in the healthcare field, in waste water treatment facilities or to treat those microorganisms that down-regulate these traits in response to QS autoinducers. Either of these types of QS modulating conjugates could be used to alter QS-controlled traits in beneficial bacteria.

In a preferred embodiment, the QS modulating conjugate described herein is formed by attaching a QS modulating molecule to a detectable moiety through a chemical bond including, but not limited to, a covalent bond. Additionally, the QS modulating conjugate can be placed in a static environment or under pressure, such as in a fluid flow environment or under controlled pressure.

In some embodiments, the QS modulating conjugate is subject to a laminar flow. In further embodiments, the flow of the fluid is characterized by a Reynolds number of less than 2000, of less than 1500, of less than 1000, of less than 750, of less than 500, of less than 400, of less than 300, of less than 200, of less than 100, of less than 50, of less than 25, of less than 10, of less than 5, of less than 4, of less than 3, of less than 2, and/or of less than 1. Other dimensions could also be used.

In some embodiments, the QS modulating conjugate is subject to a turbulent flow. In further embodiments, the flow of the fluid is characterized by a Reynolds number of greater than 2000.

In some embodiments, the QS modulating conjugate is subject to a shear stress. In further embodiments, the shear stress is characterized by a number between 0.01 and 100 Pa, between 0.01 and 90 Pa, between 0.01 and 80 Pa, between 0.01 and 70 Pa, between 0.01 and 60 Pa, between 0.01 and 50 Pa, between 0.01 and 40 Pa, between 0.01 and 30 Pa, between 0.01 and 20 Pa, between 0.01 and 10 Pa, between 0.02 and 10 Pa, between 0.03 and 10 Pa, between 0.04 and 10 Pa, between 0.05 and 10 Pa, between 0.06 and 10 Pa, between 0.07 and 10 Pa, between 0.08 and 10 Pa, between 0.09 and 10 Pa, between 0.1 and 10 Pa, between 0.02 and 100 Pa, between 0.03 and 100 Pa, between 0.04 and 100 Pa, between 0.05 and 100 Pa, between 0.06 and 100 Pa, between 0.07 and 100 Pa, between 0.08 and 100 Pa, between 0.09 and 100 Pa, between 0.1 and 100 Pa, between 0.1 and 90 Pa, between 0.1 and 80 Pa, between 0.1 and 70 Pa, between 0.1 and 60 Pa, between 0.1 and 50 Pa, between 0.1 and 40 Pa, between 0.1 and 30 Pa, between 0.1 and 20 Pa, between 0.02 and 90 Pa, between 0.03 and 80 Pa, between 0.04 and 70 Pa, between 0.05 and 60 Pa, between 0.06 and 50 Pa, between 0.07 and 40 Pa, between 0.08 and 30 Pa, and/or between 0.09 and 20 Pa. Other dimensions could also be used.

The QS modulating compound could be directly attached to the detectable moiety, and in some embodiments, any linker can be used to attach a QS modulator molecule (e.g., an antagonist or an agonist) to a detectable moiety to form a QS modulator conjugate. Examples of linkers are well known in the art, and can be synthesized in a variety of ways including, but not limited to, atom radical polymerization, reversible-addition fragmentation chain transfer polymerization, nitrous oxide-mediated polymerization, photo initiator-mediated polymerization, and can be selected based on the surface. For example, and in no way limiting, linkers can be selected from polyethylene glycol (PEGs), polyphosphazenes, polylactide, polyglycolide, polycaprolactone, poly(6-azidohexyl methacrylate), poly(2-bromoisobutyryloxyethyl methacrylate), poly(n-butyl methacrylate), poly(benzyl methacrylate), poly(cadmium methacrylate), poly(2-diethylaminoethyl methacrylate), poly(2,3-dihydroxypropyl methacrylate), poly(2-diisopropylaminoethyl methacrylate), poly(l-ethylene glycol dimethacrylate), poly(ethyl methacrylate), poly(3-ethyl-3-(methacryloyloxy methyloxetane), poly(ferrocenylmethyl methacrylate), poly(2-gluconamidoethyl methacrylate), poly(glycidyl methacrylate), poly(heptadecafluorodecyl methacrylate), poly(2-hydroxyethyl methacrylate), poly(2-hydroxylpropyl methacrylate), poly(isobutyl methacrylate), poly(isobornyl methacrylate), poly(2-lactobionamidoethyl methacrylate), poly(methacrylic acid), poly(methaacryloyladenosine), poly(3-O-methacryloly-di-Oisopropylidene-D-glucofuranose), poly(4-(10-methacryloydecyloxy)-4-pentylazobenzene), poly(2-methoxyethyl methacrylate), poly(2-(methacryloyloxy)ethyl succinate), poly(methyl methacrylate), poly(methacryloyluridine), poly(N-hydroxylsuccinimide methacrylate), poly(2-N-morpholinoethyl methacrylate), poly(octadecyl methacrylate), poly(poly(ethylene glycol) dimethacrylate, poly(poly(ethylene glycol) methacrylate, poly(poly(ethylene glycol)methyl ether methacrylate, poly(3-perylenylmethyl methacrylate, poly(2,2-dimethyl-1,3-dioxolan-4-yl methyl methacrylate), poly(sprirobenzopyran methacrylate), poly(2-(tert-butylamino)ethyl methacrylate), poly(tert-butyl methacrylate), poly(trifluoroethyl methacrylate), poly(trimethylsilyl methacrylate), poly(3-(trimethoxylsilyl)propyl methacrylate), 2-(perfluoroalkyl)ethyl methacrylate, poly(2-(1-butylimidazolium-3-yl)ethyl methacrylate hexafluorophosphate, poly(carboxybetaine methacrylate), poly(l-ethyl 3-(2-methacryloyloxy ethyl) imidazolium chloride), poly(sodium methacrylate), poly(2-methacryloyloxyethyl phosphate), poly(2-(methacryloyloxy)ethyl phosphorylcholine), poly(sulfobetanine methacrylate), poly(2-sulfatoethyl methacrylate), poly(potassium 3-sulfopropyl methacrylate), poly(acrulic acid), poly(n-butyl acrylate), poly(2-bromoacetyloxyethyl acrylate), poly(2-(2-bromopropionyloxy)ethyl acrylate), poly(benzyl acrylate), poly(11-(4-cyanophenyl-4-phenoxy)undecyl acrylate), poly(2-(dimethylamino)ethyl acrylate), poly(ethyl acrylate), poly(ethylene glycol diacrylate), poly(fluorescein acrylate), poly(1,6-hexanediol diacrylate), poly(heptadecafluorodecyl acrylate), poly(2-hydroxylethyl acrylate), poly(methyl acrylate), poly(octyl acrylate), poly(octadecyl acrylate), poly(poly(ethylene glycol)acrylate), poly(poly(glycol ethylene)acrylate succinyl fluorescein), poly(poly(glycol ethylene) methyl ether acrylate, poly(pentafluoropropyl acrylate), poly(tert-butyl acrylate), poly(trifluoroethyl acrylate), poly(trimethylsilyl acrylate), poly(triphenylamine acrylate), poly(N-(2-hydroxypropyl)methacrylamine, poly(methacrylamide), poly((3-methacryloylamino)propyl)-dimethyl-(3-sulfopropyl)ammonium hydroxide), poly(N-acryloyl glucosamine), poly(acrylamide), poly(potassium 2-acrylamido-2-methylpropane sulfonate), poly(carboxylbetane acrylamide), poly(N-cyclopropyl acrylamide), poly(N,N-dimethylacrylamide), poly(N-(3-(dimethylamino)propyl) acrylamide, poly(N-(3-dimethylamino)propyl) acrylamide methiodide), poly(N-hydroxylmethyl acrylamide), poly(N—N-methylenebisacrylamide), poly(methoxylethylacrylamide), poly(N-(6-(N-tert-butoxy-carbonylaminooxy)hexyl)acrylamide), poly(N-isopropyl acrylamide), poly(poly(ethylene glycol) methyl ether acrylamide), poly(acetoxystryrene), poly(4-chloromethylstyrene), poly(divinylbenzene), poly(4-(perfluoroalkyl)-oxymethylstyrene), poly(tert-butoxy-vinylbenzyl-polyglycidol), poly(4-methylstyrene), poly(N-octadecyl-N-(4-vinyl)-benzoyl-phenylalanineamide), polystyrene, poly(4-(poly(ethylene glycol) methyl ether styrene), poly(4-vinylaniline), poly(4-vinylbenzocyclobutene), poly(vinylquinoline), poly(4-styrenesulfonate), poly(4-vinylbenzoate), poly(1-(4-vinylbenzyl)-3-(butyl-imidazolium hexafluorophophate), poly(2-vinylpyridine), poly(3-vinylpyridine), poly(acrylonitrile), poly(itaconic acid), poly(maleic anhydride), poly(N-vinylimidazole), poly(N-vinyl-2-pyrrolidone), poly(N-vinyl-2-pyrrolidone), poly(m-isopropenyl-dimethyl-benzyl isocyanate), poly(2-vinyl-4,4-dimethyl azlactone), or any other combinations thereof. In some embodiments, combinations include any two or more of the aforementioned linkers attached at one end to a detectable moiety and at the other end to a QS modulating molecule. In other embodiments, combinations include any two or more of the aforementioned linkers arranged serially, e.g., a first linker having one end attached to a detectable moiety and another end attached to a second linker, the second linker having one end attached to the first linker and another end attached to a third linker, etc.) wherein the third linker (or ultimate linker) is attached to the QS modulating molecule. In other embodiments, any polymer architecture that consists of any combination of two or more of the aforementioned linkers including, but not limited to, end-functional linear polymers, di-end functional linear polymers, telechelic polymers, many-arm star polymers, copolymers, block polymers, dendritic polymers, branched polymers, gradient polymers, grafted polymers, microgel polymers, etc. are included. In other embodiments, linkers may not be required and the detectable moiety can be directly attached to a QS modulating molecule.

Any specific chemistry can be used to form a chemical bond between a detectable moiety and a linker and between the linker and the QS modulating molecule. Any specific chemistry can be used to form a chemical bond between a detectable moiety and a QS modulating molecule.

According to embodiments of the present invention, a variety of specific chemistries can be used to form a chemical bond between a QS modulating molecule and a detectable moiety or between a QS modulating molecule, a linker and a trackable moiety. Thus, a variety of synthetic chemical strategies are available to QS modulating conjugates for manipulation of QS. Examples of specific chemistries include, but are not limited to, biorthogonal reactions, click chemistry, thiol-ene reactions, gold-sulfide bond formation, esterification reactions, Grignard reactions, Michael reactions, ketone/hydroxylamine condensations, Staudinger ligations, strain-promoted alkyne-azide cycloadditions, photo-click cycloadditions, Diels-Alder cycloadditions, tetrazine-alkene/alkyne cycloadditions, Cu-catalyzed alkyne-azide cycloadditions, Pd-catalyzed cross coupling, strain promoted alkyne-nitrone cycloadditions, Cross-metathesis, Norbornene cycloadditions, Oxanorbornadiene cycloadditions, tetrazine ligations, tetrazole photoclick chemistry, or any other combinations of these chemistries.

Present invention embodiments also relate to a method of screening a test compound that can modulate (i.e. reduce/inhibit or promote) QS, biofilm formation, biofilm streamer formation, and/or virulence factor production by a microorganism, by contacting the test QS modulating conjugate (i.e., antagonist or agonist) with the microorganism and by measuring the modulation (i.e., reduction/elimination or promotion) of QS, biofilm formation, biofilm streamer formation, growth, and/or morphology changes. This method includes monitoring either: (1) the reduction and/or elimination of QS, biofilm formation, biofilm streamer formation, virulence factor production, growth, and/or morphology/phenotypic changes; or (2) the promotion and/or increase of QS, biofilm formation, biofilm streamer formation, virulence factor production, growth, and/or morphology/phenotypic changes. For example, by measuring the expression of a fluorescent protein, such as GFPmut2 engineered to be driven by a QS-controlled promoter and/or by measuring the expression of a second fluorescent protein such as mKate2 engineered to be driven by a constitutively expressed promoter, one can determine the ability of a test compound, e.g., a QS modulating conjugate, to inhibit or enhance QS activities by measuring fluorescence and comparing the two fluorescent protein production levels. Other embodiments including a method of screening test compounds to identify compounds that can inhibit, promote or affect biofilm and/or biofilm streamer formation are also contemplated.

Bacterial organisms may be Gram-positive or Gram-negative. Gram-positive bacteria have a peptidoglycan coating covering the bacterial cell membrane. Thus, in some embodiments, a linker can be used to join the QS modulating compound to the detectable moiety. The linker is of a sufficient length to traverse the peptidoglycan coating in order to interact with the receptor on the underlying cell membrane. In other embodiments, the QS modulating conjugate can diffuse through the peptidoglycan layer to contact receptors on the bacterial cell membrane. In some embodiments, the peptidoglycan coating may have a thickness ranging from about 1 nm to about 200 nm, from about 1 nm to about 100 nm, from about 1 nm to about 50 nm, from about 15 nm to about 50 nm, from about 15 nm to about 30 nm, or from about 1 nm to about 15 nm.

In some embodiments, the QS modulating conjugates can be provided to pre-existing biofilms. Successful administration of QS modulating conjugates will require that the compounds are delivered deep into existing biofilms, including to the substratum-biofilm interface. In one embodiment, a QS modulating conjugate was shown to diffuse into existing biofilms of a thickness of about 50 μm. In another embodiments, biofilms may have a thickness ranging from about 1 μm to about 1 cm, from about 1 μm to about 100 μm, from about 1 μm to about 50 μm, or from about 1 μm to about 10 μm.

Embodiments of the present invention also relate to a method of detecting specific microorganisms that can respond to specific QS modulating conjugates. An unknown microorganism that contacts the QS modulating conjugate will bind specifically to the conjugant, and this specific binding can be used to detect particular types of microorganisms. In one embodiment, a sample that contains an unknown bacterium that causes an infection in a patient in a healthcare setting can be contacted with a QS modulating conjugate, and if the specific binding is measured with a detectable moiety, the bacterium may be identified. In another embodiment, a sample that contains an unknown bacterium that causes contamination in a food processing setting can be introduced to the QS modulating conjugate. If specific binding is observed, using a fluorescent microscope or PET scanner, or other detectors, the bacterium may be identified. This application can be more rapid and provide lower detection limits than conventional microbial detection methodologies such as PCR verification techniques, immunological methods, and amplification methods in use today, which could be important to treat severely ill patients.

In still other embodiments, multiple types of QS modulating conjugates e.g., two or more antagonists, two or more agonists, or a combination of antagonists and agonists may be co-administered to a patient to detect multiple bacterial species.

Embodiments of the present invention also relate to a method of treating specific microorganisms that can react to specific QS modulating conjugates. An unknown microorganism that contacts the QS modulating conjugate will, in response to contact, undergo a change in QS phenotype, and this alteration can be used to treat particular types of microorganisms, for example to suppress pathogenicity and/or reduce biofilm formation in healthcare settings and/or in a food processing setting and/or in an industrial setting.

### BRIEF DESCRIPTION OF THE DRAWINGS

Embodiments are illustrated by way of example and are not limited to the figures of the accompanying drawings, in which like references indicate similar elements and in which:

FIG. 1 illustrates the Agr QS responses of S. aureus to exogenously supplied agonists and antagonists or conjugates of those compounds attached to detectable moieties. To follow the QS status of S. aureus cells, confocal microscopy and an S. aureus reporter strain that produced the fluorescent protein mKate2 in response to exogenous addition of AIP-I was used. The reporter strain was a ΔagrBDCA ΔRNAIII S. aureus mutant harboring agrCA driven by the native agrP2 promoter. Thus, the strain did not produce endogenous AIP-I or Flourescein-AIP-I but plasmid restoration of AgrC-I and AgrA endowed the strain with the capability to detect AIP-I if it was supplied exogenously. The plasmid also harbored the Agr-activated agrP3 promoter fused to mkate2. Therefore, in response to exogenously provided fluorescein-AIP-I, the reporter strain fluoresced red. This QS response was repressed by the administration of AIP-II. Thus, the reporter cells do not produce their own autoinducers. However, once the cells detected the exogenous autoinducers, the cells produced mKate2 (red fluorescent protein).

FIG. 2 illustrates a QS modulating conjugate, in this case the fluorescently tagged agonist (fluorescein-AIP-I), an agonist (AIP-1), and a fluorescent tag (fluorescein). The QS modulating conjugate retained activity for QS. Specifically, Fluorescein-AIP-I retained activity for Agr QS autoinducers (as well as the ability for fluorescence, see e.g., FIG. 3 and FIG. 4).

FIG. 3 demonstrates production of mKate2 in the reporter strain to measure QS activities. The S. aureus reporter strain was incubated in the presence of buffer only, 1 μM AIP-I (i.e., the agonist), 1 μM AIP-I (i.e., the agonist)+400 nM AIP-II (i.e., the antagonist), 1 μM Flourescein-AIP-I (i.e., the QS modulating conjugate, in this case a QS agonist conjugate), 1 μM Flourescein-AIP-I+400 nM AIP-II, and 1 μm Flourescein (a detectable moiety). mKate2 was produced only when AIP-I or Flourescein-AIP-I was supplied to the cells. When the reporter strain was simultaneously provided with 1 μM AIP-I and 400 nM AIP-II (i.e., the antagonist), little mKate2 production occurred, indicating AIP-I and Flourescein-AIP-I specifically bind to AgrC receptors. Thus, these data confirm that the hybrid molecule retains its activity for QS and it specifically reacts with QS receptors.

FIG. 4. Fluorescence measurement of AIP-I agonist, Flourescein (a trackable moiety) or Flourescein-AIP-1 (a QS modulating conjugate). Both Flourescein and Flourescein-AIP-1 show activity for fluorescence. In some embodiments, the chemical bond between the QS modulating molecule and fluorescein can be generated with a different type of chemistry, including but not limited to amide bond, triazole (click chemistry), NHS—NH₂ bonds, or another type of bond. In additional embodiments, the fluorescein molecule itself can be replaced with another type of trackable molecule such as a PET probe.

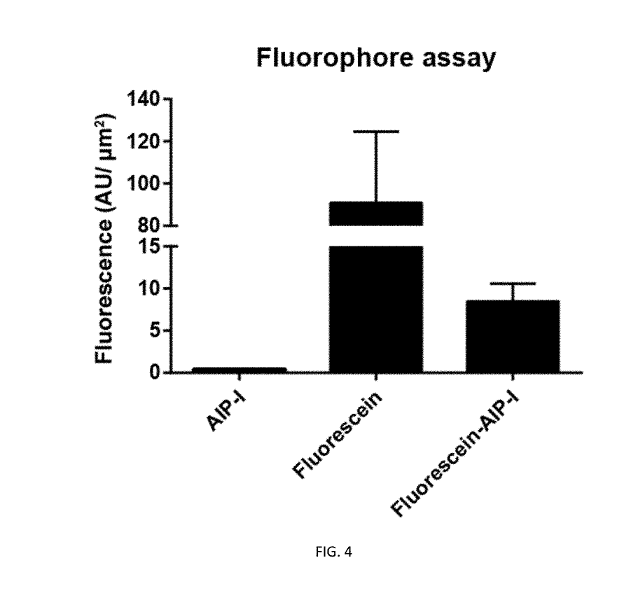

FIG. 5. Fluorescence measurement of Fluorescein-AIP-I at 30% laser power, and a Flourescein reference at 8% laser power.

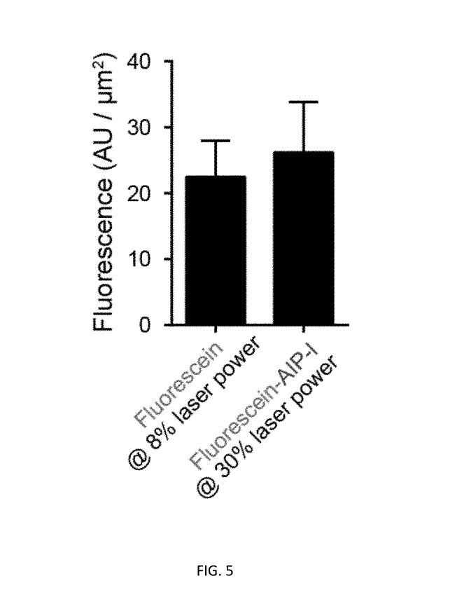

FIG. 6 shows an illustration of the experimental setup for visualizing QS modulating conjugates binding to QS receptors on living cell membranes. A microscope is used to visualize a slide containing a microfluidic chamber that was previously seeded with QS reporter cells, followed by introduction of a QS modulating conjugate such as Flourescein-AIP-1. Initially, reporter cells are exposed to 100 nM Fluorescein-AIP-I for 30 min. Then, a cell-free, molecule-free, medium was used as wash to remove unattached fluorescent molecules from the slide prior to fluorescence measurement.

FIG. 7 shows images of visualizing QS modulating conjugates binding to QS receptors, according to the protocol of FIG. 6. Here, cells that bind and respond to autoinducers (shown in green, e.g., using Flourescein-AIP-1), also express mKate2 (shown in red).

FIGS. 8A-C show images of visualizing QS modulating conjugates (e.g., Flourescein-AIP-1) binding to QS receptors along with controls. Panel 8A shows cells having AgrC⁺ receptors binding to Flourescein-AIP-1 and expressing mKate. Panel 8B shows that Flourescein alone did not bind to cells expressing AgrC⁺ receptors. Panel 8C shows that Flourescein-AIP-1 did not bind to cells lacking the AgrC (denoted as AgrC⁻) receptor. Only the cells in panel 8A expressed mKate2.

FIG. 9 shows an illustration of a microfluidics chamber, in which fluorescence from the conjugate binding to the receptors on a group of cells was measured in different locations corresponding to different time points.

FIG. 10 shows images of QS modulating conjugates binding to QS receptors on cells, corresponding to FIG. 9, wherein the green fluorescence produced by the conjugate binding to a group of cells is measured at different locations corresponding to different time points. Here, fluorescence decay was visualized as a function of time and space, with fluorescence decay arising from photobleaching (horizontal: multiple illuminations for the consecutive time points at a specific location) and diffusion (vertical: a single illumination at different time points at different locations).

FIGS. 11A and 11B show images of QS modulating conjugates unbinding to QS receptors on cells. The dissociation rate or ‘k_{off}’ (unbinding of QS modulating conjugates when treated with competitive inhibitors or in buffer) can be measured. Autoinducer dissociation and the corresponding cell responses were observed with microscopy in real time. FIG. 11A showed slow unbinding of Fluorescein-AIP-I from AgrC receptors, in an environment with buffer contained, as a function of time. FIG. 11B showed competitive inhibitors (AIP-II) facilitating the unbinding of Fluorescein-AIP-I from AgrC receptors as a function of time. In both cases, k_{off} values can be determined in situ.

FIGS. 12A and 12B show images of fluorescently tagged QS modulating conjugates binding to Agr QS receptors on S. aureus cell surfaces. In this example, the QS modulating conjugate (Flourescein-AIP-1) was used to identify the specific bacteria present, S. aureus, as AIP-1 only binds to S. aureus and not to another type of bacteria, such as P. aeruginosa. FIG. 12A shows fluorescein-AIP-I binding to S. aureus, constitutive expression of mKate, and a merged composite of both the red and green channels. FIG. 12B shows fluorescein-AIP-I did not bind to P. aeruginosa, constitutive expression of mKate, and a merged composite of both the red and green channels.

FIG. 13 shows images of QS modulating conjugates (Fluorescein-AIP-1) mixed with S. aureus and P. aeruginosa. In this example, the fluorescently tagged autoinducer (Fluorescein-AIP-1) was used to identify the specific bacteria present, in this case, S. aureus, as Fluorescein-AIP-1 only binds to Agr QS receptors on S. aureus and not to other types of QS receptors on other types of bacteria, such as P. aeruginosa. FIG. 13 shows a merged composite of fluorescein-AIP-I (green) binding to receptors on S. aureus (constitutive expression of mKate: red), not on P. aeruginosa (constitutive expression of mCherry: red), and a merged composite of both the red and green channels. In some embodiments, detection of bacteria using fluorescent probes, as discussed herein, can be faster than traditional methods of identifying bacteria, such as by PCR.

FIGS. 14A-14B show another example of using QS modulating conjugates (Fluorescein-AIP-1) to identify a pathogen. Here, Fluorescein-AIP-1 was mixed with V. cholerae and various S. aureus strains capable of Agr QS, respectively. In these examples, the fluorescently tagged autoinducer (Fluorescein-AIP-1) was used to identify S. aureus Agr-II and S. aureus Agr-III (FIG. 14B), as AIP-1 only binds to receptors on S. aureus and not different receptors on other types of bacteria, such as V. cholera (FIG. 14A). Moreover, present techniques can be used to identify the location of the pathogen. For example, if a biological sample is obtained from the gut, wherein S. aureus and multiple other types of bacteria are present, QS modulating conjugates Fluorescein-AIP-1 will specifically bind to the cell surface of S. aureus, allowing S. aureus to be detected. An identical strategy can be applied to detect other type of pathogens or specific bacteria.

FIG. 15 shows another application, in which present invention embodiments can be combined with PET probes or other detectable markers to pinpoint the location of an infection in a subject (representative images derived from the internet). For example, a fluorophore in the hybrid molecule can be replaced with a PET probe, and CT or PET technology can then be used to identify the pathogen in a subject. Here, the QS portion of the QS modulating conjugate can manipulate bacterial signaling, eventually changing bacterial behaviors, for example, to inhibit toxin production.

FIG. 16 shows an example chemical synthesis to produce a QS modulating conjugate. Peptides were generated using Fmoc-based solid-phase peptide synthesis (SPPS) on hydrazine derivatized resins followed by cleavage with trifluoroacetic acid (TFA). Hydrazide peptides 1 was oxidized with NaNO₂ and subsequently underwent MESNa (sodium 2-sulfanylethanesulfonate) thiolysis. Thioesters 2 was purified with reverse phase-high-performance liquid chromatography (RP-HPLC), and then treated with TCEP (3,3′,3″-Phosphanetriyltripropanoic acid) to remove the -StBu protecting group, and cyclized in buffer at pH=7, generating, via intermediates 3, compounds 4, which is the Alkyne-AIP-I. The black oval symbol depicts a trackable moiety. Tracking-triazole-AIP-I 5 can be produced via the copper (I) catalyzed alkyne-azide cycloaddition (CuAAC) click reaction.

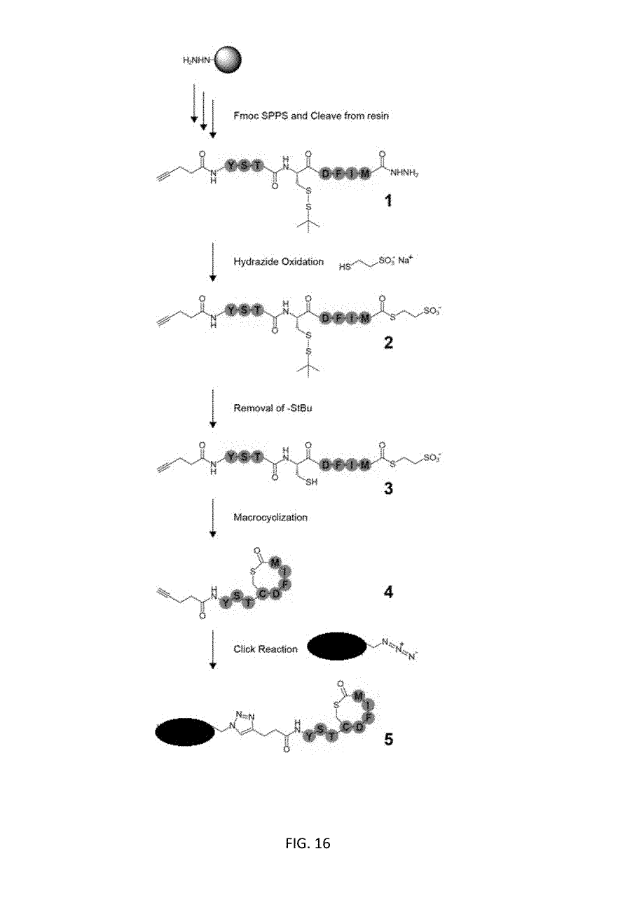

FIG. 17 shows another example chemical synthesis to produce a QS modulating conjugate. Peptides were generated using Fmoc-based solid-phase peptide synthesis (SPPS) on hydrazine derivatized resins followed by cleavage with trifluoroacetic acid (TFA). (5)6-Carboxyfluorescein was coupled to the N-terminus of the peptide. Hydrazide peptide 1 was oxidized with NaNO₂ and subsequently underwent MESNa (sodium 2-sulfanylethanesulfonate) thiolysis. Thioester 2 was purified with reverse phase-high-performance liquid chromatography (RP-HPLC), and then cyclized in buffer at pH=7, generating compound 3, which is the desired Fluorescene-AIP-I.

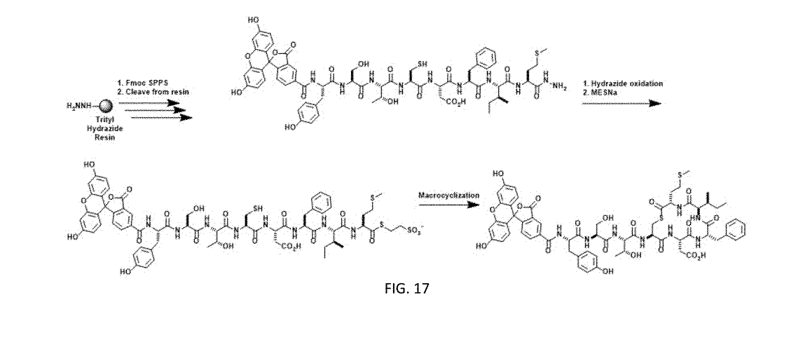

FIG. 18 shows diffusion of the compounds into thick biofilms. In this example, fluorescein-AIP-I can penetrate (shown in green) >50 μm S. aureus biofilms (shown in red). As shown in the figure, the hybrid molecules were detected at the bottom of biofilms (as represented by the yellow signal=green+red).

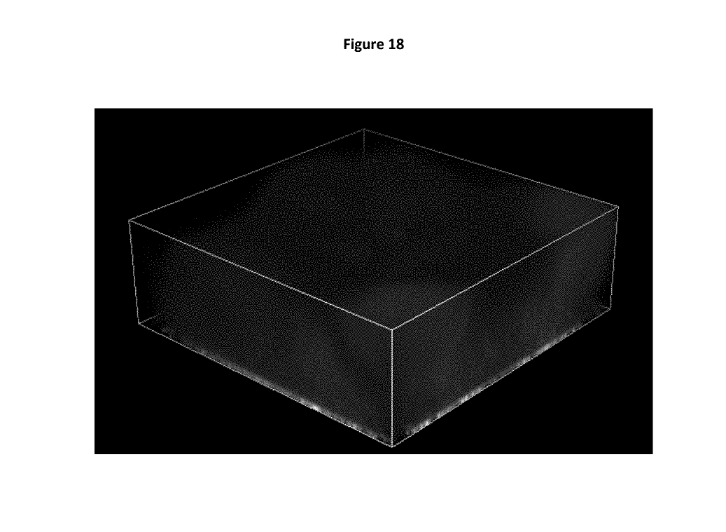

### DETAILED DESCRIPTION

### A. Definitions

Unless defined otherwise, all technical and scientific terms used herein have the same meaning as commonly understood by those of ordinary skill in the art, such as in the arts of peptide chemistry, cell culture, nucleic acid chemistry, and biochemistry. Standard techniques are used for molecular biology, genetic and biochemical methods (see, Sambrook et al., Molecular Cloning: A Laboratory Manual, 3^{rd} ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

In the present invention, a “microorganism” is defined as a bacterium, archaeon, protozoan, fungus, and/or alga.

In the present invention, “bacteria” are defined as any one of a large domain of single-celled prokaryotic microorganisms. As used herein, bacteria include any that are known to those of ordinary skill in the art and any that may be discovered. Preferred examples of bacteria are those known to be pathogenic to humans, animals or plants. Other preferred examples include those known to cause undesirable contamination and/or clogging of industrial flow systems. Still other preferred examples of bacteria include those known to infect implanted medical devices (e.g., pumps, stents, artificial joints, screws, rods, and the like). Further preferred examples of bacteria include those capable of forming biofilms and/or biostreamers or producing virulence factors. Further preferred examples include bacteria selected from the following genera: Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, and Yokenella.

Further preferred examples include bacteria selected from the following species: Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium dijficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, and Zymomonas mobilis.

Further preferred examples include bacteria selected from the class of bacteria known as Fusospirochetes.

In the present invention, “fungi” are defined as any one of a large domain of single-celled eukaryotic microorganisms such as yeasts. As used herein, fungi include any that are known to those of ordinary skill in the art and any that may be discovered. Preferred examples of fungi are those known to be pathogenic to humans, animals or plants. Other preferred examples include those known to cause undesirable contamination and/or clogging of industrial flow systems. Still other preferred examples of fungi include those known to infect implanted medical devices (e.g., pumps, stents, artificial joints, screws, rods, and the like). Further preferred examples of fungi include those capable of forming biofilms and/or biostreamers. Further preferred examples include fungi selected from the genera: Candida, Saccharomyces, and Cryptococcus.

In the present invention, an “autoinducer” is defined as a molecule that activates the expression of QS regulated genes. An “agonist” is defined as a naturally produced or synthetic autoinducer molecule that activates the expression of QS regulated genes. An “antagonist” is defined as a naturally produced or synthetic autoinducer molecule that represses the expression of QS regulated genes. Both agonists and antagonists are QS modulating molecules. An agonist or antagonist conjugated to a trackable moiety is a QS modulating conjugate.

In the present invention, “biofilms” are defined as sessile microorganism community, such as a bacterial and/or fungal communities, that occupies a surface. These biofilms can cause chronic and medical device-associated infections, clogging, and/or device failure. Biofilms are surface-associated assemblies of microorganisms, such as bacteria and/or fungi which are bound together by extracellular polymeric substances (4, 5). Biofilms are attached to the surface all along the edges, including the bottom edge, of the surface. Although bacterial biofilms are desirable in waste-water treatment (6), biofilms primarily cause undesirable effects such as chronic infections or clogging of industrial flow systems (1-3). Cells in biofilms display many behavioral differences from planktonic cells, such as a 1,000-fold increase in tolerance to antibiotics (7, 8), an altered transcriptome (9-11), and spatially heterogeneous metabolic activity (12, 13). Some of these physiological peculiarities of biofilm-dwelling cells may be due to strong gradients of nutrients and metabolites, which also affect biofilm morphology and composition (14, 15).

In the present invention, “biofilm streamers” are defined as biofilms that have been partially detached from the surface upon which the biofilm is growing. Under conditions of flow in the presence of available biofilm promotion element(s) (e.g., curves, corners, bends, etc.), the flow partially detaches the extra cellular matrix off of the substrate along with cells that were in it already and is suspended in the liquid attached only at its edges. The detached biofilm forms filaments or streamers in the flowing liquid. The streamer is then able to capture other flowing debris and cells in order to continue growing. Thus, biofilms grow by cellular division, while biofilm streamers grow both by cell division as well as cellular capture of passing cells in the flow.

In the present invention, “biofilm growth” is defined as the expansion of the surface-attached biofilm over time, whether through cell division or through attachment of additional cells to the surface from the surrounding environment. As used herein, this growth includes expansion laterally over available surfaces as well as expansion through thickening of the biofilm layer by layers of additional cells.

In the present invention, “biofilm morphology” is defined as the physical composition or shape of the biofilm. As used in the invention, biofilm morphology may change over time. These changes may be in the composition of the extracellular matrix, in the composition of microorganisms, such as bacteria and/or fungi in the biofilm, or in the shape of the biofilm. Biofilm growth would be an example of a change in biofilm morphology. Another example of a change in biofilm morphology would be the flow induced formation of biofilm streamers. A third example would be the inclusion or expulsion of different microbial species within the biofilm.

In the present invention, “biofilm streamer growth” is defined as the expansion of the biofilm streamer over time. As used herein, this expansion may be in the length of the biofilm streamer filaments and/or in the thickness of the biofilm streamer. This growth may be through cell division and/or through capture of additional cells, extracellular matrix, and/or debris from the surrounding liquid.

In the present invention, “biofilm streamer morphology” is defined as the physical composition and/or shape of the biofilm streamer. As used in the invention, biofilm streamer morphology may change over time. These changes may be in the extracellular matrix, in the composition of the microorganisms (e.g., bacteria and/or fungi) in the biofilm streamer and/or in the shape of the biofilm streamer. Biofilm streamer growth, flow induced formation and/or inclusion/exclusion of different microbial species are all examples of a change in biofilm streamer morphology.

In the present invention, “QS modulator molecule” is any molecule that modulates QS, and in turn, alters any QS phenotype including, but not limited to, a biofilm, a biofilm streamer, and/or a virulence factor. The QS modulator molecule can be an antagonist or an agonist.

In the present invention, “QS modulator conjugate” is defined as a QS modulator molecule that is attached to a trackable moiety. The QS modulator conjugate may modulate QS, and in turn, alter any QS phenotype. The QS modulator conjugate may allow detection and identification of a particular species of bacteria or other microorganism. The QS modulator conjugate retains activity both for quorum sensing and as a fluorophore. As an example demonstrated herein, Fluorescein-AIP-I retains activity as an S. aureus Agr QS autoinducer and the ability to fluorescence.

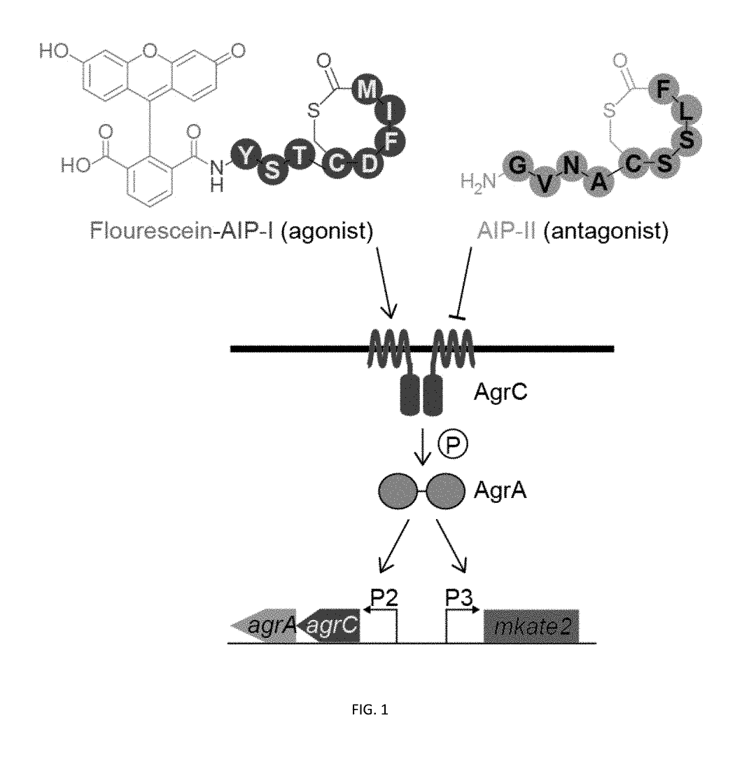

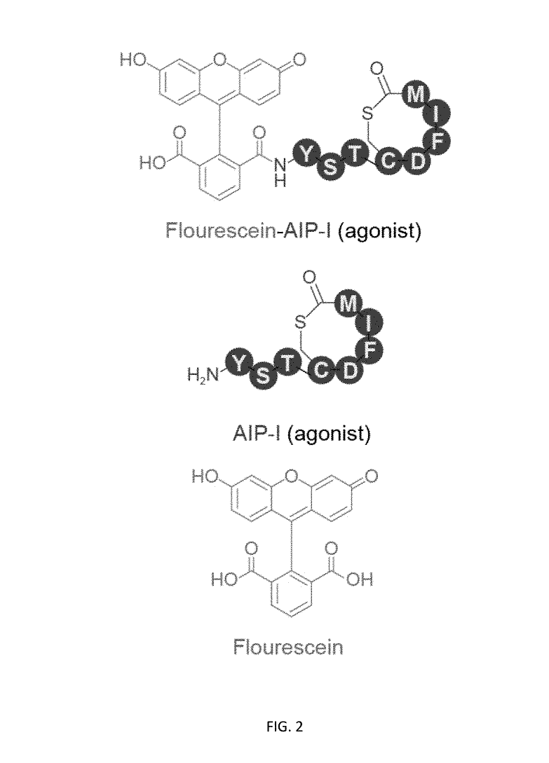

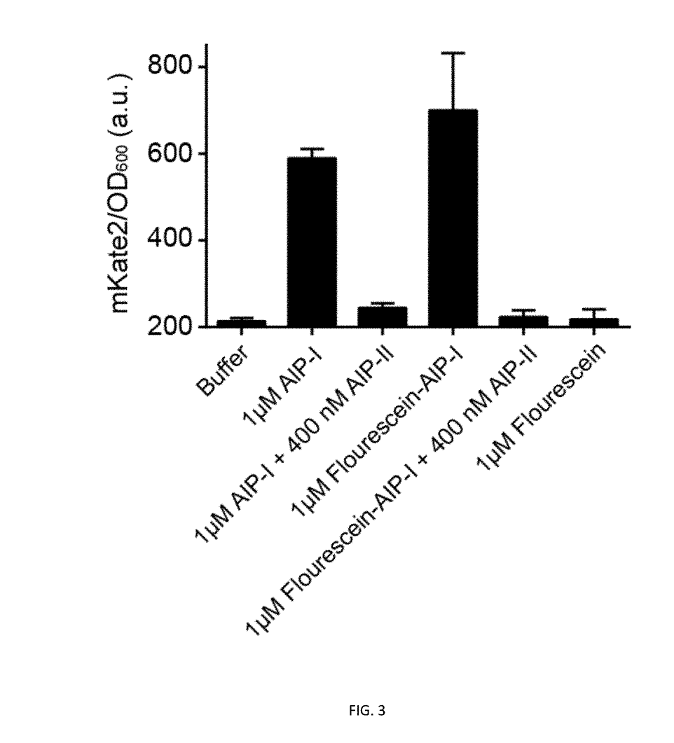

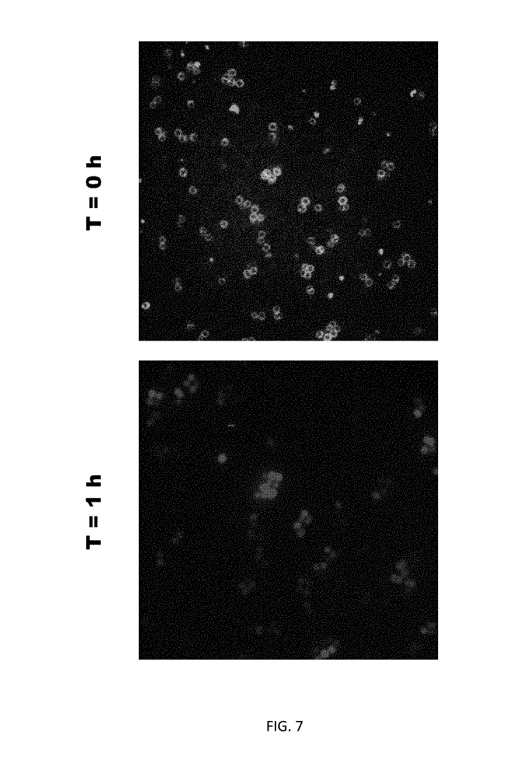

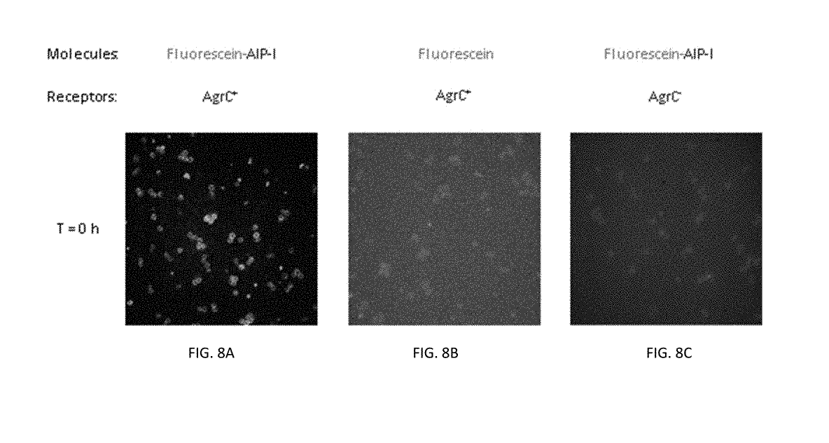

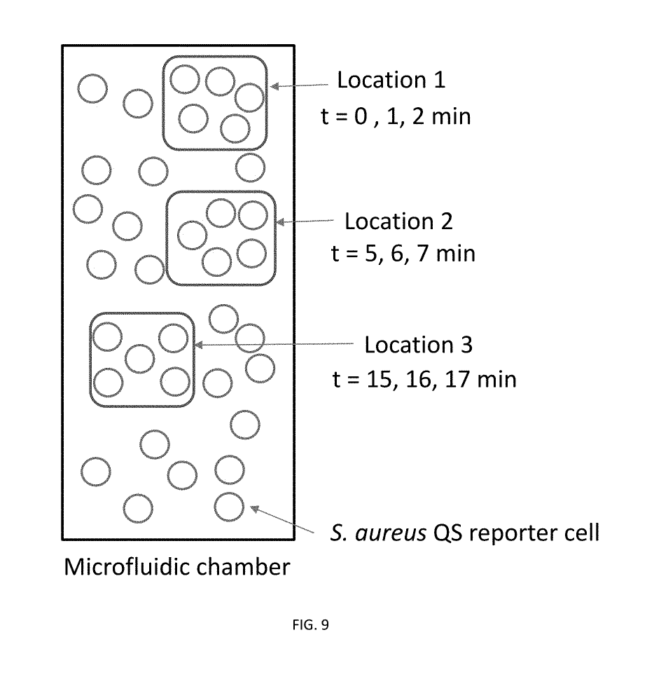

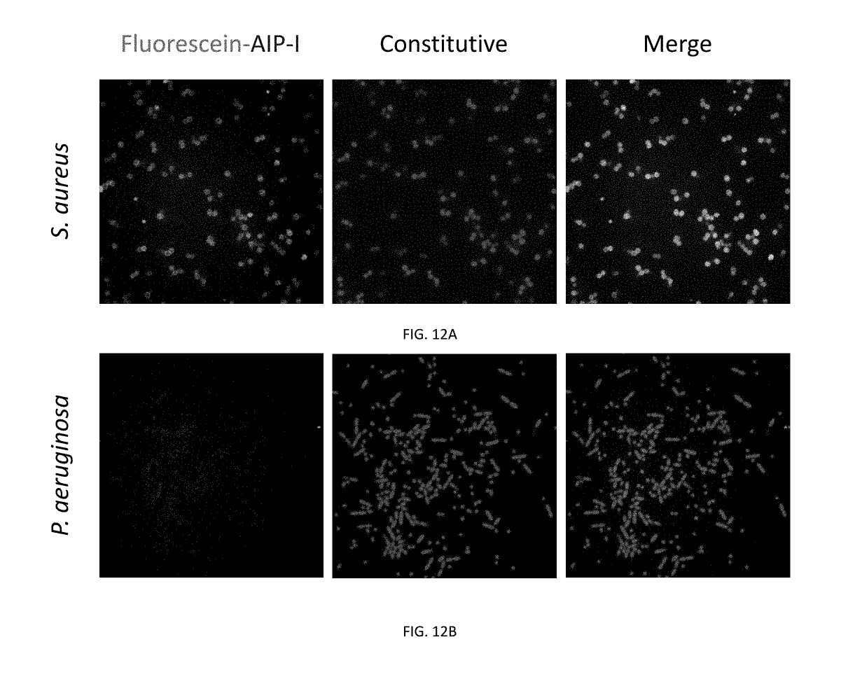

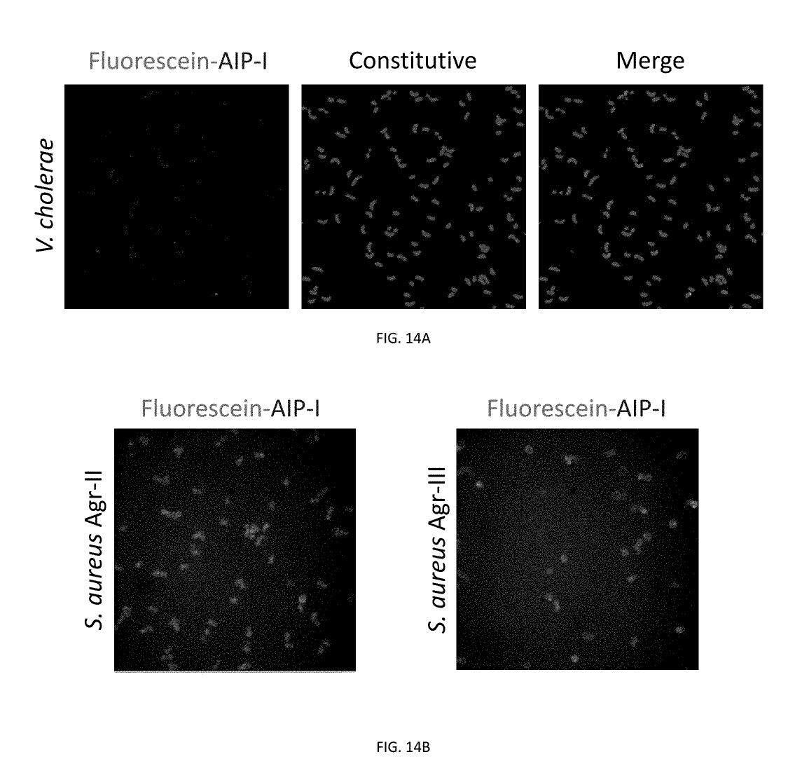

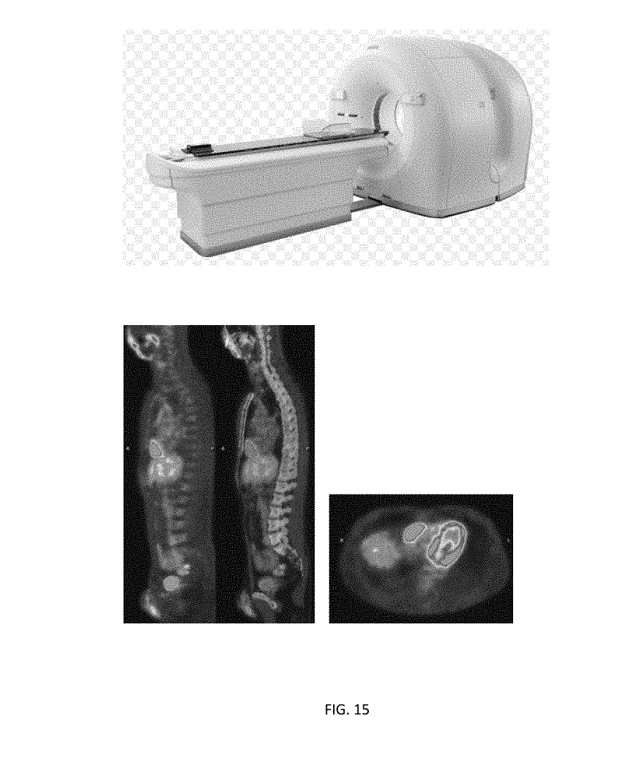

As used herein, a “QS antagonist conjugate” is defined as a molecule that antagonizes (e.g., inhibits or reduces) QS and is attached to a trackable moiety. A QS antagonist conjugate in turn, can alter any QS phenotype including, but not limited to, a biofilm, a biofilm streamer, and/or a virulence factor production and can be detected using imaging technology. Examples of QS antagonists are described in U.S. Pat. No. 8,247,443, U.S. Pat. No. 8,568,756, or PCT/US14/56497 which are specifically incorporated by reference in their entirety. See, for example, the structures described in FIGS. 2, 8 and 9 of U.S. Pat. No. 8,247,443, FIGS. 3A-P, 4A, 8A-8L and 10A-B of U.S. Pat. No. 8,568,756, and in Tables 1-4 and FIGS. 1, 6, 7, 12-15 of PCT/US14/56497, all of which are herein incorporated by reference in their entirety. Additionally, other preferred examples of QS antagonists include, but are not limited to small organic molecules, peptides and synthetic molecules. Any of these molecules can be conjugated to a detectable moiety.

Alternatively, a “QS agonist conjugate” is defined as a molecule that agonizes (e.g., promotes or induces) QS and is attached to a trackable moiety. A QS agonist conjugate in turn, can alter any QS phenotype including, but not limited to, a biofilm, a biofilm streamer, and/or a virulence factor production and can be detected using imaging technology. Examples of QS agonists are described in U.S. Pat. No. 5,353,689 and PCT/US2014/051648 both of which are incorporated by reference in their entirety. Any of these molecules can be conjugated to a detectable moiety.

As used herein, “radiolabel” refers to a technique for including radionuclides in a chemical compound, allowing the chemical compound to be tracked by imaging. Examples of radionuclides include but are not limited to ¹⁴C, ¹⁵N, ³H, ³⁵S, ³²P, ³³P, ¹²⁵I. PET probes may include radionuclides for visualization.

As used herein, “imaging” refers to technology used to visualize detectable or trackable moieties, including but not limited to microscopy, optical imaging, X-ray radiography, magnetic resonance imaging, nuclear imaging (PET-CT scans, SPECT, etc.). Imaging includes the in vivo or in vitro visualization and characterization of biological processes at the molecular and cellular level.

In the present invention, “a trackable moiety” or “detectable moiety” refers to a moiety that is detectable or trackable using imaging technology or other types of detection technologies. Trackable or detectable moieties include fluorescent tags (also known as fluorophores or fluorescent molecules, including but not limited to GFP, RFP, YFP, etc.), and radionuclides.

As used herein, “QS phenotype” or “morphology” or “trait” refers to any change in the bacterial colony/organism or in the constituents in the cells in the colony, including but not limited to, changes in appearance, e.g., an increase in streamer formation, a decrease in streamer formation, an increase in biofilm density, a decrease in biofilm density, etc. as well as other changes e.g., a change in gene expression, a change in mRNA production, a change in protein production, etc.

As used herein, “click chemistry” is a term to describe reactions that are high yielding, broadly applicable, create only byproducts that can be removed without chromatography, are stereospecific and generally simple to perform, and can be conducted in easily removable or benign solvents. In some embodiments, click chemistry allows generation of large libraries of compounds for screening in research. In one example, click chemistry enables covalent bond formation between molecule A with an azide group and with molecule B with an alkyne group. Click chemistry uses Cu catalysts to form triazoles by cycloaddition. A trackable molecule with an azide at one end may be reacted with another group (a pro- or anti-QS molecule) with an alkyne group attached at one end. Other methods are also possible, for example, the trackable moiety can have an alkyne group attached, and the QS modulating molecules can possess azide groups at one end.

Thus, the inhibition of QS, biofilm, biofilm streamer, and/or a virulence factor production and/or morphology/phenotypic changes through the use of a QS agonist/antagonist may lead to either a decrease or an increase in overall virulence to the host depending on whether the microorganism relies on QS, biofilm, biofilm streamer, and/or a virulence factor production to promote infection. Similarly, the promotion of QS, biofilm, biofilm streamer, and/or a virulence factor production and/or morphology/phenotypic changes through the use of a QS agonist/antagonist may lead to either a decrease or an increase in overall virulence to the host depending on whether the microorganism relies on QS, biofilm, biofilm streamer, and/or a virulence factor production to promote infection.

In the present invention, “fluid” is defined as a liquid or a gas. In one example, the fluid is water, with or without the addition of other components. These additional components may include, but are not limited to nutrients and salts needed to support bacterial growth, bacteria, chemical or biochemical probes to assist with visualization of cells or extracellular components, test compounds, and compounds for selective growth of specific bacterial strains. In other embodiments, a fluid is a biological fluid such as, for example, blood.

In the present invention, “flow” or “fluid flow” is defined as movement of the fluid along a surface in a continuous stream.

In the present invention, “flow rate” is defined as the volume of a fluid moving along a surface per unit time.

In the present invention, “Reynolds number” is defined as a dimensionless quantity used to help predict similar flow patterns in different fluid flow situations. It is defined as the ratio of inertial forces to viscous forces and thus quantifies the relative importance of these two types of forces for given flow conditions. Reynolds numbers may be used to characterize different flow regimes within a similar fluid, such as laminar or turbulent flow. When a fluid is flowing through a surface, such as a closed channel such as a pipe or between two flat plates, either of two types of flow may occur depending on the velocity of the fluid: laminar flow or turbulent flow. Laminar flow tends to occur at lower velocities, below a threshold at which it becomes turbulent. A Reynolds number of less than 2320 is characteristic of laminar flow in a circular tube. A Reynolds number greater than 2320 is characteristic of turbulent flow in a circular tube.

In the present invention, “laminar flow” in a long straight surface is defined as a flow regime that occurs when a fluid flows in parallel layers, with no disruption between the layers. At low velocities, the fluid tends to flow without lateral mixing, and adjacent layers slide past one another like playing cards. For flow in a long straight surface, such as a long straight channel, there are no cross-currents perpendicular to the direction of flow, nor eddies or swirls of fluids. In laminar flow, the motion of the particles of the fluid is very orderly with all particles moving in straight lines parallel to the pipe walls. For flows in more complicated geometries, such as channels with bends and corners, the laminar flow is the time-independent motion for a steady pressure drop; the flow may be three-dimensional, i.e. the velocity may have all three components non-zero, but the flow remains steady (time independent) so long as the pressure drop is constant.

In the present invention, “turbulent flow” is defined as a flow regime characterized by chaotic property changes. This includes low momentum diffusion, high momentum convection, and rapid variation of pressure and velocity in space and time. In turbulent flow, unsteady vortices appear on many scales and interact with each other. Drag due to boundary layer friction increases. The structure and location of boundary layer separation often changes, sometimes resulting in a reduction of overall drag.

In the present invention, “shear stress” is defined as the force/area acting tangent to a surface. In an ordinary fluid such as water the shear stress is proportional to the fluid viscosity and proportional to the velocity gradient (as defined in standard textbooks).

In the present invention, “controlled pressure” is defined as pressure applied to a fluid moving through a channel such that the pressure drop along the channel is held constant. Thus, as resistance to flow in the pipe is increased, rather than continuing to apply increasing pressure to keep the flow rate constant, the flow rate is reduced such that the pressure remains constant. As used herein, a constant pressure includes pressure that varies. For example, the pressure may “pulse” at a given frequency, for example, but the average pressure will remain constant.

In the present invention, “test compound” is defined as any compound added to the test system for evaluation of its effect on QS, biofilm formation, biofilm streamer formation, and/or a virulence factor production. The effect of the test compound may be to inhibit (an antagonist) or to enhance (an agonist) QS, biofilm, biofilm streamer, and/or a virulence factor production and/or morphology changes. The inhibition of QS, biofilm, biofilm streamer, and/or a virulence factor production and/or morphology changes through the use of a QS antagonist may lead to either a decrease or an increase in overall virulence to the host depending on whether the microorganism relies on QS, biofilm, biofilm streamer, and/or a virulence factor production to promote infection. Similarly, the promotion of QS, biofilm, biofilm streamer, and/or a virulence factor production and/or morphology changes through the use of a QS agonist may lead to either a decrease or an increase in overall virulence to the host depending on whether the microorganism relies on QS, biofilm, biofilm streamer, and/or a virulence factor production to promote infection. In the case of a test QS modulating conjugate, this type of compound refers to a test compound conjugated to a trackable moiety so that the test compound can be visualized using an imaging technique.

These compounds may be pharmaceutical compound, small molecules, or biological compounds. Some examples include peptides, proteins, peptidomimetics, antibodies, non-antibody specific binding molecules, such as adnectins, affibodies, avimers, anticalins, tetranectins, DARPins, mTCRs, engineered Kunitz-type inhibitors, nucleic acid aptamers and spiegelmers, peptide aptamers and cyclic and bicyclic peptides and small synthetic or natural organic molecules (Ruigrok et al. Biochem J. (2011) 436, 1-13; Gebauer et al., Curr Opin Chem Biol. (2009) (3):245-55.)

In the present invention, “antibody” refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. Examples of molecules which are described by the term “antibody” in this application include, but are not limited to: single chain Fvs, and fragments comprising or alternatively consisting of, either a VL or a VH domain. The term “single chain Fv” or “scFv” as used herein refers to a polypeptide comprising a VL domain of an antibody linked to a VH domain of an antibody. Antibodies of the invention include, but are not limited to, monoclonal, multispecific, human or chimeric antibodies or antibodies made in animals, single chain antibodies, Fab fragments, F9ab′) fragments, antiidiotypic (anti-Id) antibodies (including, e.g., anti-Idantibodies to antibodies of the invention), and epitope-binding fragments of any of the above. The immunoglobulin molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, “Gram-positive” refers to a type of bacteria surrounded by a thick layer of peptidologlycan. Gram-positive bacteria include staphylococci (“staph”), streptococci (“strep”), pneumococci, and the bacterium responsible for diphtheria (Cornynebacterium diphtheriae) as well as anthrax (Bacillus anthracis). Gram-positive bacteria react with the Gram stain to turn dark blue or violet.

As used herein, “Gram-negative” refers to bacteria that have a cytoplasmic membrane, a thin peptidoglycan layer, and an outer membrane containing lipopolysaccharide. Gram-negative bacteria do not react with the Gram stain to turn dark blue or violet, instead these bacteria appear red or pink due to counterstain (usually safranin).

As used herein, “peptidoglycan layer” refers to an elastic polymeric mesh-like network found outside the bacterial cell membrane.

As used herein, “linker length” means the longitudinal length of the linker, usually in nm.

As used herein, “linker diameter” means the diameter or breadth of the linker

As used herein, “permeability agent” means any agent capable of forming holes in the outer membrane layer of gram-negative bacteria. Examples include holins, endolysins, or bacteriocins⁵⁰.

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

### B. Agr OS

S. aureus Agr QS is driven by an autoinducer peptide (called AIP) harboring a thiolactone ring and an exocyclic tail at the N-terminus. AIP is processed from the precursor peptide AgrD by AgrB and other proteases, and the AIP is secreted^{26,27}. Extracellular AIP is detected by a cognate transmembrane-bound receptor histidine kinase, AgrC, that upon AIP binding, autophosphorylates and subsequently funnels a phosphoryl group to the partner response regulator, AgrA^{28,29} (FIG. 1). Phospho-AgrA activates the agrP3 promoter driving transcription of RNAIII that has multiple roles³⁰. RNAIII functions as an mRNA that encodes δ-toxin (a membrane disrupting exo-protein that lyses eukaryotic host cells), and RNAIII also participates in regulation of other genes required for exo-toxin secretion and biofilm disassembly³¹. Detection of AIP launches the autoinduction positive feedback loop that increases AIP production, resulting in amplification of the QS response⁸.

There are four S. aureus Agr allelic variants (I to IV) that make four AIPs differing only in a few amino acid residues. AIPs activate QS in the S. aureus cells that produce them, and they generally inhibit QS in heterologous S. aureus cells possessing different AIP variants⁸. In the S. aureus agr-I strain referenced herein, AIP-I is the native autoinducer.

TrAIP-II is a universal inhibitor for S. aureus Agr QS systems³². TrAIP-II competes with the cognate AIPs for binding to the receptor³². Unless otherwise indicated, AIP-I acts as an autoinducer agonist and TrAIP-II or AIP-II acts as a competitive antagonist to S. aureus agr-I (FIG. 1a). S. aureus agr-I was selected for study because it possesses the most prevalent Agr type found world-wide in nosocomial infections.

According to present invention embodiments, AIP-I is conjugated to Flourescein to form Flourescein-AIP-I. The fluorescent tag allows visualization of AIP-I binding to its cognate receptor, e.g., on the surface of S. aureus.

### C. Uses of OS Modulators

Present invention embodiments include applications for any natural, industrial, or biomedical area where the presence of S. aureus or any other microorganism could be detrimental. Present invention embodiments have immediate applications as a health-care tool in S. aureus infection and other pathogenic settings. This technology has the capability to differentiate one species of bacterium (S. aureus) against other species within a shorter time than any other identification method. Also, present invention embodiments allow monitoring of the binding of the QS modulating conjugate to its cognate receptor, including associated characteristics such as diffusion rate, location in a biofilm in situ, location in an infection site or patient sample in vivo or ex vivo, and wherein the visualization is performed in real time or near real time.

The QS portion of the QS modulator conjugate affects the QS regulatory network, thereby reducing the severity of pathogenesis of the microorganism. Furthermore, pro- and anti-QS molecules are not prone to bacterial antibiotic-resistance, leading to improved treatment for the bacterial infections. In addition, this technology can be expanded to a broad range of other pathogens that use QS pathways to control virulence, including but not limited to, Pseudomonas aeruginosa, Vibrio cholerae, Staphylococcus epidermidis, Streptococcus pneumonia, Streptococcus mutans, Streptococcus sanguinis, and Enterococcus facaelis. Thus, this technology has potential therapeutic and diagnostic applications for many infections that S. aureus and other pathogens cause.

According to embodiments of the present invention, the QS modulating conjugate is formed by conjugating QS modulating molecules to trackable moieties. The conjugation chemistry can be diverse, including but not limited to click chemistry. Other chemistries could likewise be used to produce QS modulating conjugates. The present technology can also be used with any trackable moieties, including radionuclides, fluorophores, PET probes, etc. The present technology can also be used to deliver a toxin or other molecules (such as, for example, a payload) that can be used to kill the bacteria. In this manner, any known molecule having antibacterial activity can be targeted using the compositions described herein. The present technology can also be used with any bacterial species possessing a QS system.

The compositions described herein, e.g., the QS modulating conjugate, can be used to modulate QS, biofilm formation, biofilm streamer formation, and/or virulence factor production or any other QS-controlled trait of interest. The compositions described herein can also be used to identify particular types of microorganisms based upon binding to their cognate receptor. For example, present invention embodiments include generating a QS modulating conjugate and visualizing binding of this conjugate to the microorganism, e.g., a receptor such as AgrC on an S. aureus cell. In one embodiment, the QS modulating conjugate may be used to detect a particular microorganism among a variety of microorganisms. For instance, according to the examples presented herein, a fluorescently tagged agonist (Fluorescein-AIP-1) may be used to detect the presence of S. aureus. Fluorescein-AIP-1 binds specifically to AgrC, which is expressed in S. aureus and not other types of microorganisms, allowing S. aureus to be detected. In some embodiments, detection can occur in vitro, e.g., from a biological sample obtained from a patient, from a sample obtained from an industrial or medical or food production facility, etc. In other embodiments, detection can occur in vivo, e.g., using techniques to detect a QS modulating conjugate within a patient. In some embodiments, the QS modulating conjugate can be used to determine the presence of one particular type of microorganism. In other embodiments, multiple types of QS modulating conjugates can be co-administered to a patient or contacted with a biological sample to detect multiple species of microorganisms. Here, each type of QS modulating molecule (e.g., an agonist specific to a S. aureus, a different agonist specific to P. aeroginsa, etc.) would have a distinct trackable moiety (e.g., AIP-1 may be tagged with green fluorescent molecules, 3-oxo-C12 homoserine lactone which is specific to the P. aeruginosa Las QS system would be tagged with red fluorescent molecules, etc.) for their corresponding detection. For in vivo applications, each type of QS modulating molecule (e.g., an agonist specific to a S. aureus, a different agonist specific to P. aeruginosa, etc.) would have a distinct trackable moiety. In a further embodiment, present techniques may be used to determine the efficacy of an anti-microbial treatment, e.g., by monitoring the presence of the microorganism as a function of time, the efficacy of an antibiotic or other treatment can be assessed. If the presence of the microorganism decreases as a function of time, then the treatment (alone or in combination with the patient's immune system) is assumed to be effective in controlling the infection. The QS modulating conjugate has applications for any natural or artificial environment in which the presence of a microorganism, such as, for example, S. aureus could be detrimental. The QS modulating conjugate described herein allows for detection of pathogenic microorganisms.

In still other embodiments, the methods of screening for agonists or antagonists of QS, biofilm formation, biofilm streamer formation, and/or virulence factor production can be performed by fluorescent tagging a test compound, e.g., a test QS modulating conjugate, and determining if the test compound binds to the microorganism. These screens may additionally be run in the presence of various antibiotics to detect effectors that enhance antibiotic inhibition.

The QS modulating conjugate can be used to positively and negatively manipulate QS in bacteria such as S. aureus. For example, QS agonist conjugates or QS antagonist conjugates can successfully be used to control bacterial QS. In S. aureus, Agr QS activation leads to the production of a battery of virulence factors that are responsible for invasion and dissemination in host tissues¹⁰. Agr QS in S. aureus also activates the biofilm disassembly process⁴². Thus, precisely manipulating Agr QS using synthetic strategies can terminate virulence while not enabling biofilm formation. Due to resistance of S. aureus, there is an urgent medical need for the control of S. aureus.

QS modulating conjugates can be used in scenarios such as acute infections, e.g. staphylococcal scalded skin syndrome and toxic shock syndrome where it is essential to halt production of exo-toxins. S. aureus cells residing in biofilms are more resistant to antibiotics and host immune defenses than are their planktonic counterparts⁴³. AIP-I and/or AIP-I conjugates, by triggering biofilm dispersal and transitioning the S. aureus cells to the planktonic lifestyle, can render the dispersing cells more susceptible to antibiotics and to host immune defenses.

The techniques provided herein, which use pro- or anti-QS molecules attached to a trackable moeity to detect microorganisms are not limited to S. aureus. These strategies are generally applicable to any bacterial strain. For example, the Gram-positive bacterium Enterococcus faecalis causes life-threatening urinary tract infections, bacteremia, endocarditis, and meningitis in humans⁴⁴. Pathogenicity of E. faecalis relies on the Fsr QS system, which is homologus to the S. aureus Agr QS system⁴⁵. However, importantly, in the case of E. faecalis, activation of Fsr QS promotes both biofilm formation and virulence factor production^{45,46}. Thus, a trackable moiety harboring Fsr QS antagonists (i.e., ZBzl-YAA5911⁴⁷) can have multiple benefits in identifying the bacterium, preventing biofilms, and reducing exo-toxin production. Such multiple benefits can also be imagined for other bacteria such as Listeria monocytogenes and Streptococcus pyogenes, as both pathogens possess Agr-type QS systems that activate biofilm formation and virulence factor expression at high cell density⁴⁸. Moreover, the beneficial bacterium Lactobacillus plantarum, which is important in the dairy and fermented food industries, also has an Agr-type QS system called Lam that could be manipulated in applications in food production⁴⁹. Thus, any bacterial system capable of QS can be modulated and the presence of the bacteria detected based on the techniques provided herein.

Clearly, the techniques provided herein have the potential to be expanded to other systems with known ligands and with accessible cognate receptors. Present invention embodiments are not intended to be limited to the examples provided herein, and may be applicable in any bacterial system (or non-bacterial system) which utilizes QS.

Administration of the QS modulating conjugate to a subject or a patient in need thereof is specifically contemplated. For example, a QS antagonist conjugate can be administered to a patient to detect an infection as well as determine whether a treatment as a function of time is effective against the infection.

Blocking virulence is one of the strategies contemplated to combat these bacteria. This approach provides less selective pressure for the spread of resistant mutants and leads to drug therapies that are effective over a greater time span compared to traditional antibiotics. Rather than preventing growth or killing the bacteria, an antivirulence approach prevents the expression of virulence traits. The bacteria that have been treated and are thus benign should then be more easily cleared by the host immune system.

Examples of QS modulating molecules are shown in Tables 1A-1B. Any of the following molecules can be conjugated to a detectable moiety such as a fluorescent or radioactive label. Each of the references in Table 1A are included herein in their entirety.

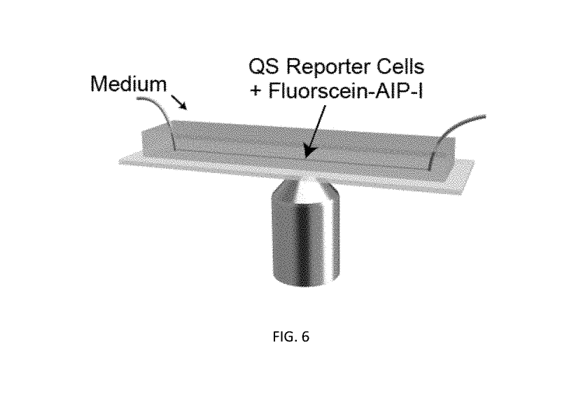

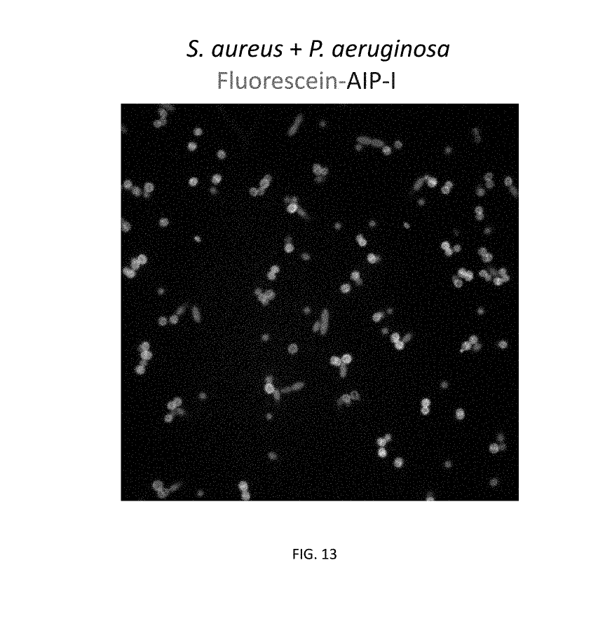

TABLE 1A

| Pat. No. | Reference | Compounds |
|---|---|---|
| U.S. Application 7,419,954 | Table 1 | — |
| | General formula | — |
| | from col. 7 line 53 | |
| | to col. 8, line 11 | |
| | FIGs. 1A and 6 | — |
| U.S. Application 6,953,833 | General formula | — |
| | from col. 4 lines | |
| | 43 to 67 | |
| | FIG. 1A | — |
| PCT/US2014/056497 | Table 1 | Entry 1-12 |
| | Table 2 | Entry 1-23 |
| | Table 3 | Entry 1-23 |
| | Table 4 | Entry 1-20 |
| | General formulas | — |
| | shown in paragraphs | |
| | [0011], [0012], | |
| | [0013] and [0015] | |
| U.S. Pat. No. 8,568,756 | FIG. 3 | Antagonist 6807-0002 |
| (same antagonists | | Antagonist 8008-8157 |
| also in U.S. Pat. No. | | Antagonist Cl04-0038 |
| 8,772,331 and | | Antagonist Cl05-2488 |
| U.S. Pat. No. 8,247,443) | | Antagonist 3448-8396 |
| | | Antagonist 3578-0898 |
| | | Antagonist 3643-3503 |
| | | Antagonist 4052-1355 |
| | | Antagonist 4248-0174 |
| | | Antagonist 4401-0054 |
| | | Antagonist 4606-4237 |
| | | Antagonist C137-0541 |
| | | Antagonist C450-0730 |
| | | Antagonist C540-0010 |
| | | Antagonist C646-0078 |
| U.S. Pat. No. 8,247,443 | General formula | — |
| | shown in col. 7, | |
| | lines 1-33 | |
| U.S. Pat. No. 8,535,689 | FIGs. 13a-13e | Compounds 1-33, |
| | | and CAI-1 |
| WO 2014/092751 | FIG. 2A | Compounds 1-11 |
| | FIG. 3 | Compounds 11-18 |

Unless otherwise indicated, the compounds of PCT/US2014/056497 and U.S. Pat. No. 8,568,756 function as antagonists of QS to inhibit the QS pathway.

It is also expressly understood that the compounds referred to (and incorporated by reference) in PCT/US2014/056497 are limited to those that exhibit anti-pathogenic and anti-biofilm activity through inhibition of QS.

Unless otherwise indicated, the compounds of U.S. Pat. No. 8,535,689 and WO 2014/092751 function as agonists. Some QS systems, such as those found in cholera, work “in reverse” from other QS systems. For example, agonists of cholera QS receptors repress biofilm formation and pathogenicity, effectively functioning as inhibitors of bacterial infections.

Other molecules, including flavonoid compounds, function as antagonists of QS, and are shown in Table 1B.

TABLE 1B

| Compound No. | Chemical Name | Structure |
|---|---|---|
| #3 | Chrysin | 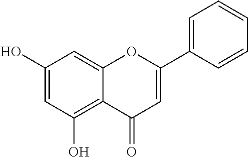 [SMILES: O=c1cc(-c2ccccc2)oc2cc(O)cc(O)c12] |
| #43 | Apigenin | 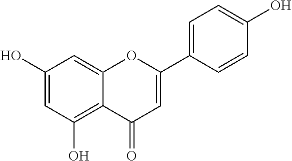 [SMILES: O=c1cc(-c2ccc(O)cc2)oc2cc(O)cc(O)c12] |
| #48 | Quercetin | 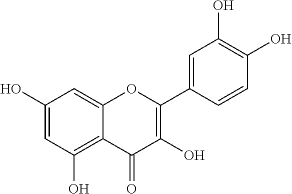 [SMILES: O=c1c(O)c(-c2ccc(O)c(O)c2)oc2cc(O)cc(O)c12] |
| #46 | Baicalein | 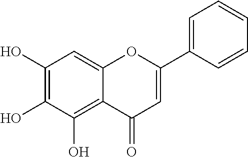 [SMILES: O=c1cc(-c2ccccc2)oc2cc(O)c(O)c(O)c12] |
| #54 | 7,8-dihydroxyflavone | 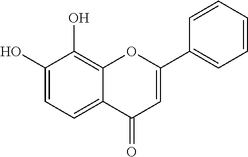 [SMILES: O=c1cc(-c2ccccc2)oc2c(O)c(O)ccc12] |
| #53 | 6-dihydroxyflavone | 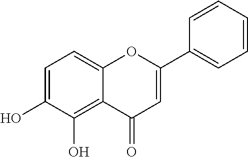 [SMILES: O=c1cc(-c2ccccc2)oc2ccc(O)c(O)c12] |
| #4 | Narigenin | 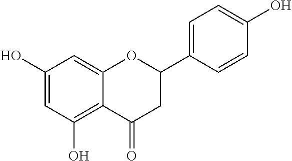 [SMILES: O=C1CC(c2ccc(O)cc2)Oc2cc(O)cc(O)c21] |
| #1 | Phloretin | 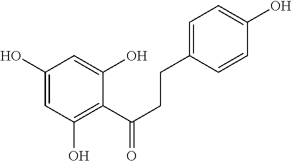 [SMILES: O=C(CCc1ccc(O)cc1)c1c(O)cc(O)cc1O] |
| #18 | 3,5,7-trihydroxyflavone | 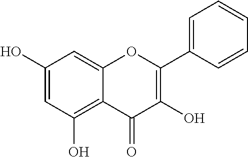 [SMILES: O=c1c(O)c(-c2ccccc2)oc2cc(O)cc(O)c12] |
| #19 | Pinocembrin | 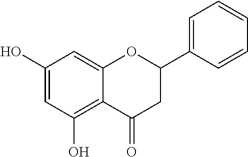 [SMILES: O=C1CC(c2ccccc2)Oc2cc(O)cc(O)c21] |

It is expressly understood that present invention embodiments include both agonists and antagonists. In some systems, compounds act as antagonists with respect to the QS system to repress biofilm formation and pathogenicity, while in other systems, compounds act as agonists with respect to the QS system to repress biofilm formation and pathogenicity.

### EXAMPLES

### Example 1: Generation of a OS Modulating Conjugate

Peptides were generated using Fmoc-based solid-phase peptide synthesis (SPPS) on hydrazine derivatized resins followed by cleavage with trifluoroacetic acid (TFA). (5)6-Carboxyfluorescein was coupled to the N-terminus of the peptide. Hydrazide peptide 1 was oxidized with NaNO₂ and subsequently underwent MESNa (sodium 2-sulfanylethanesulfonate) thiolysis. Thioester 2 was purified with reverse phase-high-performance liquid chromatography (RP-HPLC), and then cyclized in buffer at pH=7, generating compound 3, which is the desired Fluorescene-AIP-I.

Peptides were generated using Fmoc-based solid-phase peptide synthesis (SPPS) on hydrazine derivatized resins followed by cleavage with trifluoroacetic acid (TFA), see FIG. 13. Hydrazide peptides 1 were oxidized with NaNO₂ and subsequently underwent MESNa (sodium 2-sulfanylethanesulfonate) thiolysis. Thioesters 2 was purified with reverse phase-high-performance liquid chromatography (RP-HPLC), and then treated with TCEP (3,3′,3″-Phosphanetriyltripropanoic acid) to remove the -StBu protecting group, and cyclized in buffer at pH=7, generating, via intermediates 3, compounds 4, which is the Alkyne-AIP-I. The black oval symbol depicts a trackable moiety. Tracking-triazole-AIP-I 5 can be produced via the copper (I) catalyzed alkyne-azide cycloaddition (CuAAC) click reaction.

Any specific chemistry can be used to make a chemical bond between a QS modulating molecule and a trackable moiety. For example, a bioorthogonal reaction can be used. This reaction is highly selective and has no side reactions. The chemistry is biocompatible and thus not toxic to living organisms, and the fast kinetic reactions make this process especially convenient. Many reported bioorthogonal reactions are known in the art and can be used to conjugate tags to QS-modulating molecules, such as Staudinger Ligation and/or click chemistry. Additional reactions that are known in the art and that can be used include, but are not limited to: nitrone dipole cycloaddition, norbornene cycloaddition, tetrazine ligation, and/or quadricyclane ligation.

Any autoinducers as described herein (agonist or antagonist) can be used for conjugation to the surfaces. As a proof of principle, S. aureus autoinducer (AIP) peptides were used, but the chemical reaction is identical for other QS modulating molecules. Present invention embodiments can be used with other fluorophores, other type of tracking moieties, other types of QS molecules, and other species of bacteria.

### Example 2: Methods for Measuring OS, Biofilm Production, Biofilm Streamer Production and/or Virulence Factor Production

Methods for measuring QS, biofilm production, biofilm streamer production and/or virulence factor production have been reported in the literature and are herein incorporated by reference in their entirety. Kim M K et al. “Local and global consequences of flow on bacterial quorum sensing,” Nature Microbiology 1:15005 2016; Kim M K et al. “Filaments in curved streamlines: Rapid formation of Staphylococcus aureus biofilm streamers,” New J Phys. 2014 Jun. 26; 16(6):065024; Ng W L, et al., “Broad spectrum pro-quorum-sensing molecules as inhibitors of virulence in vibrios,” PLoS Pathog. 2012; 8(6); and O'Loughlin C T, “A Quorum-Sensing Inhibitor Blocks Pseudomonas Aeruginosa Virulence And Biofilm Formation,” PNAS (2013) October 29; 110(44):17981-6. Compositions described herein can be tested in any of these published protocols.

### Example 3: Methods for Measuring OS, Biofilm Production, Biofilm Streamer Production and/or Virulence Factor Production

QS Measurements

QS measurements at the transcriptional level were assessed using promoter fusion analysis. Promoters driving genes responsible for QS (e.g., in the case of P. aeruginosa, the lasI and rhlI promoters were used. In the case of V. cholerae, the qrr4 and/or luxC promoters were used. In the case of S. aureus, the agrP3 promoters were used to measure QS activities. In all cases, the promoters were fused to genes encoding fluorescent proteins, luciferase, or the beta-lactamase enzyme, and/or an equivalent which can be quantitatively measured temporally and spatially using a microscopy or a spectrometer. Other promoters and/or reporter proteins could readily be used.

QS phenotypes are diverse, but in the context of healthcare settings, measuring pathogenic traits that are regulated by QS systems, such as virulence factor production and biofilm formation, are of interest. The following example assays may be used to quantitatively measure such traits.

Virulence Factor Production Measurements

Virulence factor production at the transcriptional level is assessed using promoter fusion analysis. Promoters driving genes responsible for virulence factors, e.g., in the case of P. aeruginosa, the lasAB and rhlAB promoters were used. In the case of V. cholerae, the ctxAB, toxT and hapA promoters were used. In the case of S. aureus, the hldBC and clfB promoters were used. In all cases, the promoters were fused to genes encoding fluorescent proteins, luciferase, or the beta-lactamase enzyme, or an equivalent which can be quantitatively measured temporally and spatially using microscopy or a spectrometer.

The actual virulence factor (toxin, enzymes, etc.) can also be measured directly. Specifically, one can measure or verify the results from promoter-reporter fusions using enzyme-linked immunosorbent assay (ELISA) techniques, in which toxins from a sample are transferred to a membrane, and subsequently, antibodies that recognize the specific toxin are introduced. The antibodies are usually linked to an enzyme or a fluorophore that can be quantitatively measured.

Biofilm Production Analysis

One can measure the amount of biofilms formed using cells carrying a constitutively expressed fluorescent protein. Microscopy can be used to measure the 3D volumes or biomass.

Biofilms can also be measured using a conventional method. There are many commercially available stains that specifically bind to components of biofilms, such as the polysaccharide matrix and/or extracellular DNA. Subsequently, using microscopy, the amount of biomass can be quantified. Biofilms can also be measured in a commonly used microtiter plate assay and crystal violet staining.

### Example 4: Construction of Bacterial Strains and Plasmids

The strains and plasmids used are listed in Table 4. Staphylococcus aureus strains include RN4220, RN9011, RN6390b, RN6911, and RN6607. Plasmids include pJL1111 and pRN7062. S. aureus strains MK121¹⁸ (RN6390b carrying pMK021; agrP3-gfpmut2, sarAP1-mkate2) and S. aureus MRSA strain MK131¹⁸ (BAA1680 carrying pMK021) were also used.

TABLE 4

| | | |
|---|---|---|
| | | References/ |
| Strain/plasmid | Genotype/description | Sources |

E. coli

| | | |
|---|---|---|
| DH5α | Cloning strain, F′ proA⁺B⁺ lacI^{q} Δ(lacZ)M15 zzf:Tn10 (Tet^{R})/ | NEB |
| | fhuA2Δ(argF-lacZ)U169 phoA glnV44 Φ80Δ(lacZ)M15 gyrA96 | |
| | recA1 relA1 endA1 thi-1 hsdR17 | |

S. aureus

| | | |
|---|---|---|
| RN4220 | Restriction-deficient mutant of strain 8325-4, transformable | S1 |
| | cloning host | |
| RN9011 | RN4220 containing pRN7023 (SaPI-1 integrase) | S2 |
| RN6390b | Standard agr-I wild-type, derivative of NTCT8325-4 | S3 |
| RN6911 | RN6390b replacing agrBDCA and RNAIII with tetM (ΔagrBDCA | S4 |
| | ΔRNAIII) | |
| RN6607 | Standard agr-II wild-type | S6 |
| MK231 | RN6911 SaPI-1attC::pMK031 (sarAP1-mturquoise2 in the | This study |
| | genome) | |
| MK232 | RN6911 SaPI-1attC::pMK032 (sarAP1-gfpmut2 in the genome) | This study |
| MK233 | RN6911 SaPI-1attC::pMK033 (sarAP1-mko in the genome) | This study |
| MK241 | MK231 containing pMK051 | This study |
| MK242 | MK232 containing pMK051 | This study |
| MK243 | MK233 containing pMK051 | This study |
| MK244 | MK232 containing pMK014 | This study |
| MK245 | MK232 containing pMK004 | This study |
| MK260 | RN6911 SaPI-1attC::pMK060 (agrP2-agrCA in the genome) | This study |
| MK261 | MK264 containing pMK004 | This study |
| MK264 | RN6911 SaPI-1attC::pMK064 (agrP2-agrCA, agrP3-mkate2 in | This study |
| | the genome) | |
| MK265 | MK264 containing pMK012 | This study |
| MK121 | RN6390b containing pMK021; agrP3-gfpmut2, sarAP1-mkate2 | S5 |
| | Note that pMK021 does not contain agrP2-agrCA. | |
| MK131 | Methicillin resistant strain (MRSA), clinical isolate from human | S5 |
| | skin, agr group I strain containing pMK021; agrP3-gfpmut2, | |
| | sarAP1-mkate2 | |
| MK125 | RN6390b containing pMK013; sarAP1-mko | This study |
| MK126 | RN6607 containing pMK013; sarAP1-mko | This study |

Plasmids

| | | |
|---|---|---|
| pMK004 | pCN54 (Erm^{r}) containing agrP3-mkate2 | S5 |
| pMK011 | pCN54 (Erm^{r}) containing sarAP1-mturquoise2 | This study |
| pMK012 | pCN54 (Erm^{r}) containing sarAP1-gfpmut2 | This study |
| pMK013 | pCN54 (Erm^{r}) containing sarAP1-mko | This study |
| pMK014 | pCN54 (Erm^{r}) containing sarAP1-mkate2 | S5 |
| pRN7062 | pCN54 (Erm^{r}) containing agrP2-agrCA, agrP3-lacZ | S6 |
| pMK051 | pCN54 (Erm^{r}) containing agrP2-agrCA, agrP3-mkate2 | This study |
| pJC1111 | SaPI-1 attS suicide vector containing cadmium resistant (cadCA) | S7 |
| pMK031 | pJC1111 (Cad^{r}) containing sarAP1-mturquoise2 | This study |
| pMK032 | pJC1111 (Cad^{r}) containing sarAP1-gfpmut2 | This study |
| pMK033 | pJC1111 (Cad^{r}) containing sarAP1-mko | This study |
| pMK060 | pJC1111 (Cad^{r}) containing agrP2-agrCA | This study |
| pMK064 | pJC1111 (Cad^{r}) containing agrP2-agrCA, agrP3-mkate2 | This study |

DNA polymerase, dNTPs, and restriction enzymes were purchased from New England Biolabs (NEB, Ipswich, Mass.). DNA extraction and purification kits were acquired from Qiagen (Valencia, Calif.). DNA oligonucleotides were purchased from Integrated DNA Technologies (Coralville, Iowa). Sequences of plasmids were verified by Genewiz (South Plainfield, N.J.).

Plasmids carrying constitutively expressed fluorescent fusions were constructed by replacing the mkate2 gene from pMK014 (sarAP1-mkate2) with genes encoding different fluorescent proteins (gfpmut2, mturquoise2, and mko). To make these plasmids, the gfpmut2 gene was amplified by PCR from pMK021¹⁸ using primers MKF013/MKR013, the mturquoise2 gene⁵¹ was amplified by PCR from pDP428 using primers MKF011/MKR011 and the mko gene was amplified by PCR from pCN005⁵² using primers MKF014/MKR014. mkate2 was replaced with an amplified gene by overlap extension PCR cloning. These plasmids were called pMK012 (sarAP1-gfpmut2), pMK011 (sarAP1-mturquoise2), and pMK013 (sarAP1-mko).

The constitutively expressed reporter fusions were integrated into the S. aureus chromosome using a site-specific integration suicide vector, pJC1111, carrying a cadmium resistance cassette and a SaPI-1 attS sequence which integrates into the S. aureus chromosomal attachment site (attC) of pathogenicity island 1 (SaPI-1)⁵³. This plasmid was integrated in single copy and maintained stably⁵³. pJC1111 was digested using restriction enzymes NarI/SphI. The sarAP1-gfpmut2 gene was amplified by PCR from pMK012 using primers MKF031/MKR031 followed by digestion with NarI/SphI and ligation into digested pJC1111. This plasmid was called pMK032 (sarAP1-gfpmut2 in the suicide vector). The same procedure was used for other fluorescent genes, pMK031 (sarAP1-mturquoise2 in the suicide vector), and pMK033 (sarAP1-mko in the suicide vector). The plasmids were introduced into Escherichia coli DH5α using chemical transformation (New England Biolabs, Ipswich, Mass.) followed by selection with ampicillin. The plasmids were purified from E. coli, introduced by electroporation into S. aureus strain RN9011, which expresses the SaPI-1 integrase, and colonies containing the fusions integrated into the chromosome were selected with cadmium. Subsequently, the chromosomal integrants were transduced into S. aureus strain RN6911 using standard phage transduction techniques with phage 80α. These strains were called MK232 (sarAP1-gfpmut2 in the genome), MK231 (sarAP1-mturquoise2 in the genome), and MK233 (sarAP1-mko in the genome). Primers are shown in Table 5.

TABLE 5

| Primers | Sequence (5′-3′) |
|---|---|
| MKF011 | TCGTTAACTAATTAATTTAAGAAGGAGATATACATATGGTATCAAAAGGGGA |
| | AGAGTTG (SEQ ID NO: 1) |
| MKR011 | TTAGAATAGGCGCGCCTTATTTGTACAGTTCGTCCATGCC (SEQ ID NO: 2) |
| MKF013 | TCGTTAACTAATTAATTTAAGAAGGAGATATACATATGAGTAAAGGAGAAGA |
| | ACTTTTCACT (SEQ ID NO: 3) |
| MKR013 | TTAGAATAGGCGCGCCTTATTATTTGTATAGTTCATCCATGCCATG (SEQ ID |
| | NO: 4) |
| MKF014 | TCGTTAACTAATTAATTTAAGAAGGAGATATACATATGGTGAGTGTGATTAA |
| | ACCAGAG (SEQ ID NO: 5) |
| MKR014 | TTAGAATAGGCGCGCCTTAGGAATGAGCTACTGCATCTTCTA (SEQ ID NO: 6) |
| MKF031 | TATAATAGCATGCACATAACACCAAAAAGAAGAAGGTGC (SEQ ID NO: 7) |
| MKR031 | CCGCAAAGGCGCCTGTCACTTTGCTTGATATATGAG (SEQ ID NO: 8) |
| MKF032 | AATACGCCGTTAACTGACTTTATTATCTTATTATATTTTTTTAACGTTTCTCAC |
| | CGATGC (SEQ ID NO: 9) |
| MKR032 | GGAGGGGCTCACGACCATACTTACATGTCAACGATAATACAAAATATAATAC |
| | AAAATATA (SEQ ID NO: 10) |
| MKF033 | TTGAATACGCCGTTAACTGACTTTATTATCTTATTATATTTTTTTAACGTTTCT |
| | CACCGA (SEQ ID NO: 11) |
| MKR033 | GGAGGGGCTCACGACCATACTTA (SEQ ID NO: 12) |

A plasmid carrying a transcriptional fusion to monitor S. aureus Agr QS activity was constructed by replacing the lacZ gene from vector pRN7062⁵⁴ (agrP3-lacZ) with the mkate2 gene. pRN7062 also harbored the genes encoding the Agr QS detection components agrCA under their native agrP2 promoter but driven in the opposite direction. To make this plasmid, the lacZ gene was removed from pRN7062 by digestion with EcoRI/NarI. The mkate2 gene was obtained from pMK014 by EcoRI/NarI digestion. The digested mkate2 gene was ligated into digested pRN7062. This plasmid was called pMK051 (agrP2-agrCA, agrP3-mkate2). This construct was first introduced into E. coli, purified, and subsequently introduced into S. aureus strain RN4220 using selection with erythromycin. Subsequently, using phage transduction, the plasmid was introduced into S. aureus strains MK232, MK231, and MK233. The resultant strains were called MK242 (sarAP1-gfpmut2 in the genome and pMK051), MK241 (sarAP1-mtor2 in the genome and pMK051), MK243 (sarAP1-mko in the genome and pMK051). To construct the S. aureus AagrBDCA strain harboring agrP3-mkate2 in a plasmid, the vector pMK004¹⁸ (agrP3-mkate2) was introduced into S. aureus strain MK232 (sarAP1-gfpmut2 in the genome of S. aureus ΔagrBDCA), leading to strain MK245.

Control strains were constructed to study heterogeneity of the Agr QS response. The first control strain had the agrP2-agrCA and agrP3-mkate2 genes inserted into the genome of RN6911, and harbored sarAP1-gfpmut2 in a plasmid. To make this strain, the agrP2-agrCA, and agrP3-mkate2 genes from pMK051 were amplified using primers MKF032/MKR032, and this fragment was inserted into the suicide vector pJC1111 by overlap extension PCR cloning. This plasmid was called pMK064 (agrP2-agrCA, agrP3-mkate2 in the suicide vector). The gene was integrated into the S. aureus strain RN6911 chromosome as described above. This strain was called MK264 (agrP2-agrCA, agrP3-mkate2 in the genome). The vector pMK012 (sarAP1-gfpmut2) was introduced into MK264, leading to strain MK265 (agrP2-agrCA, agrP3-mkate2 in the genome and pMK012). The second control strain was constructed by introducing pMK014 (sarAP1-mkate2) into strain MK232, leading to MK244 (sarAP1-gfpmut2 in the genome and pMK014). The third control strain had the agrP2-agrCA gene inserted into the genome of RN6911, and harbored agrP3-mkate2 in a plasmid. To construct this strain, the agrP2-agrCA gene from pMK051 was amplified using primers MKF033/MKR033, this fragment was inserted into the suicide vector pJC1111 by overlap extension PCR cloning. This plasmid was called pMK060 (agrP2-agrCA in the suicide vector). The gene was integrated into the S. aureus strain RN6911 chromosome as described above. This strain was called MK260 (agrP2-agrCA in the genome). The vector pMK004¹⁸ (agrP3-mkate2) was introduced into MK260, leading to strain MK261 (agrP2-agrCA in the genome and pMK004). Finally, a constitutively expressed mKO fluorescent reporter (sarAP1-mko in pMK013) into wild-type S. aureus agr-I (strain RN6390b) and wild-type S. aureus agr-II (strain RN6607) was used to measure the number of cells in biofilms on surfaces.

### Example 5: Growth Conditions

S. aureus RN6911 derivatives were grown overnight at 37° C. with shaking in Tryptic Soy Broth (TSB; Difco, Franklin Lakes, N.J.) with 10 μg/ml tetracycline and 10 μg/ml erythromycin to maintain plasmids, back-diluted 1:200, and re-grown for 3 h (to OD₆₀₀˜0.05-0.1). S. aureus MK121, MK131, MK125, and MK126 were grown overnight at 37° C. with shaking in TSB with 10 μg/ml erythromycin, back-diluted 1:2000, and re-grown for 3 h (to OD₆₀₀˜0.05-0.1).

### Example 6: Synthesis of AIP-I, AIP-II and Derivatives

AIP-I, AIP-II and derivatives were synthesized using a combined solid-phase/solution-phase approach. Linear peptide □-thioester precursors were generated using Fmoc-solid phase peptide synthesis employing a hydrazine linker system. The peptides were then cyclized in solution to create the thiolactone macrocyclic.

### Example 7: Fluorescence Reporter Assay

Transcription from fluorescence reporter genes was measured in S. aureus strain MK242. Overnight cultures were diluted 1:200 into fresh TSB with 10 μg/ml tetracycline and 10 μg/ml erythromycin, re-grown, and 90 μl of these cultures were distributed into wells of 96 well plates (MatTek, Ashland, Mass.), followed by addition of 10 μl of AIP-I and/or Fluorescein AIP-II and/or derivatives. Subsequently, 50 μl of mineral oil was added (Sigma, St. Louis, Mo.) to prevent evaporation. Using a Synergy 2 plate reader (Biotek, Winooski, Vt.), GFPmut2 and mKate2 levels were measured at 484 nm/528 nm and 588 nm/633 nm, respectively. Measurements were conducted with 15 min intervals at 37° C. with shaking.

### Example 8: Microscopy and Imaging

Imaging was performed using a Nikon Eclipse Ti inverted microscope (Melville, N.Y.) fitted with a Yokogawa CSU X-1 confocal spinning disk scanning unit (Biovision Technologies, Exton, Pa.) and DU-897 X-9351 camera (Andor, Concord, Mass.). Laser lines at 445, 488, 543, and 592 nm were used to excite the mTurquoise2, GFPmut2, mKO, and mKate2 fluorescent proteins, respectively. Laser lines at 488, 543, and 592 nm were used to excite Alexa Fluor 488 fluorophore, Alexa Fluor 555 fluorophore, and Alexa Fluor 594 fluorophore, respectively. In order to obtain single-cell resolution, both a 100× oil objective with N.A. 1.4 (Nikon, Melville, N.Y.) and a 1.5× lens placed between the CSU X-1 and the Nikon microscope side port were used. Consequently, the magnification of 0.1 μm per pixel in the XY plane was obtained. For single-cell analysis, custom code was written in Matlab. Briefly, the area of an individual cell was recognized and segmented using a watershed-based algorithm. In this process, cells were removed if they were on the edge of the image or if they were smaller than 30% of the average cell size, suggesting that they were out of focus. In the area of an individual cell, both the constitutive GFPmut2 fluorescence and the QS controlled mKate2 fluorescence were measured, subtracted from background signals and summed. The normalized QS output was calculated as the QS controlled mKate2 intensity divided by the constitutively expressed GFPmut2 intensity in individual cells. In each experiment, images of many regions on the surfaces were taken to include 1000-4000 individual cells. Each replicate was performed using independent bacterial cultures and independent surfaces at room temperature. Identical procedures were performed for the strains harboring different constitutive fluorescent proteins such as mTurquoise2 and mKO. Custom code was used to count the cells in the biofilms. Each image was segmented in the z-plane and assessed independently.

### Example 9: Quantification of Autoinducers

We can calculate the number of autoinducers that bind to a cell. We can do this by measuring the flourescence output from the bound hybrid molecules that bind to receptors in vivo in real time. Subsequently, the total fluorescence output is subtracted from the background signal, and divided by the average single-molecule intensity. This method yields the number of hydrid molecules that are attached to a cell surface, which corresponds to the total number of binding sites (i.e., number of receptors) on a cell surface when the attached hydrid molecules are satuarating.

### Example 10: Measurement of kₒₙ/k_{off}

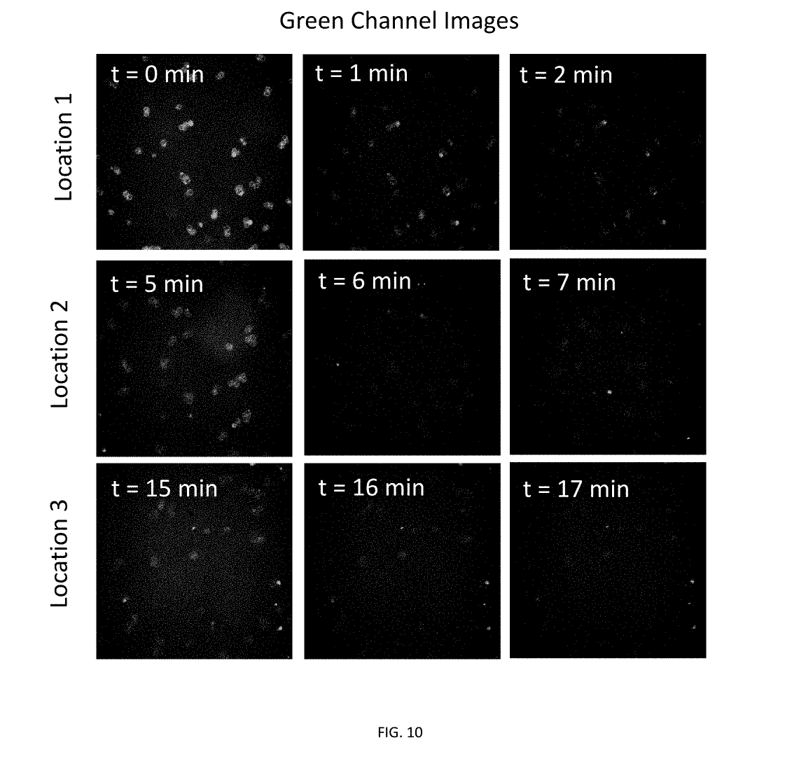

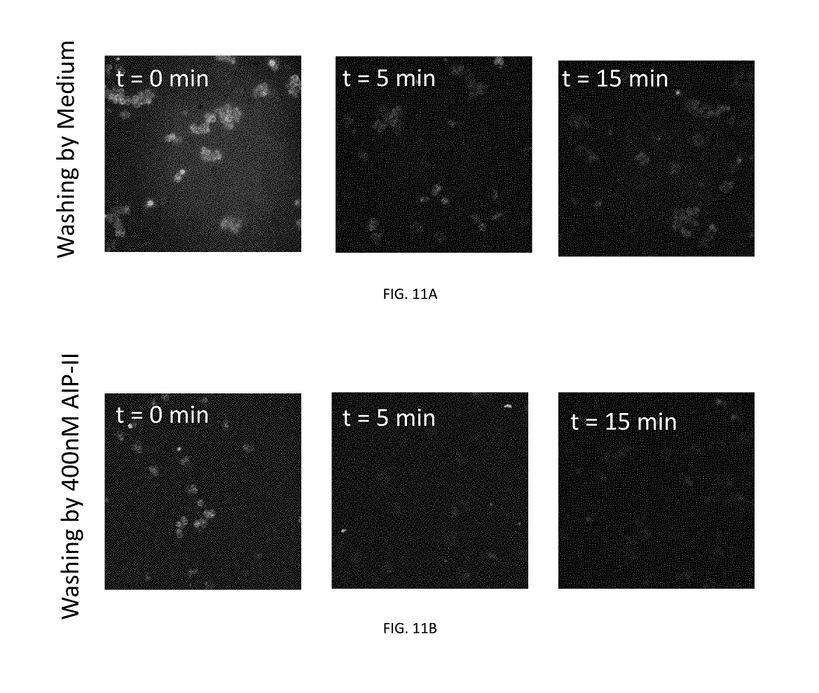

We can measure kinetic constants such as kₒₙ/k_{off} (the rates of binding and unbinding of autoinducers) in vivo in real time. As indicated in FIGS. 10 and 11, diffusion of attached molecules from QS receptors was observed over time. As indicated in Example 9, we can measure the number of attached molecules on a cell surface over time, which can in turn yield the dissociation constant. An modified strategy can be applied as follows to ascertain the association constant: Initially no hydrid molecules are attached to cells, and subsequently, hydrid molecules are provided to cells over time. And, the number of attached molecules were quantified over time, which can in turn gives the association constant.

### Example 11: Measurement of Diffusion Constants

Diffusion constants of hybrid molecules to pre-existing biofilms can be measured in situ. As indicated in Example 9, we can measure the number of attached hybrid molecules in three-dimensional biofilms over time, which can provide the diffusion coefficients of the molecules into biofilms. The diffusion coefficient can provide information about the material properties of specific biofilms.

### Example 12: Location of Bacterial Colonization

We can prove the biomedical and industrial applications of the hybrid molecule in realistic settings, for example that require pinpointing of bacterial colonization. In realistic settings, such as the human body or mouse models of infection that S. aureus has colonized, the QS conjugate harboring a trackable moiety is provided. Using appropriate imaging or other appropriate detection technology to detect the trackable moiety, we can pinpoint the location of bacterial colonization.

### Example 13: Manipulation of Bacterial Colonization

We can prove the biomedical and industrial applications of the hybrid molecule to manipulate bacterial behaviors in realistic settings, for example biofilm formation or toxin production. In realistic settings, such as in a human host, or an industrial pipe, or in food or in a mouse model of infection that S. aureus has colonized, a QS conjugate harboring a trackable moiety is provided. Subsequently, the degree of alteration in QS-controlled activities and/or QS-directed phenotypes such as biofilm formation, toxin production or colonization are measured.

### REFERENCES

1 Wang, B. & Muir, T. W. Regulation of virulence in Staphylococcus aureus: molecular mechanisms and remaining puzzles. Cell Chem Biol 23, 214-224 (2016).
2 Kong, K. F., Vuong, C. & Otto, M. Staphylococcus quorum sensing in biofilm formation and infection. Int J Med Microbiol 296, 133-139 (2006).
3 Drescher, K. et al. Architectural transitions in Vibrio cholerae biofilms at single-cell resolution. Proc Natl Acad Sci USA 113, 2066-2072 (2016).
4 Yan, J., Sharo, A. G., Stone, H. A., Wingreen, N. S. & Bassler, B. L. Vibrio cholerae biofilm growth program and architecture revealed by single-cell live imaging. Proc Natl Acad Sci USA 113, 5337-5343 (2016).
5 Nadell, C. D., Drescher, K. & Foster, K. R. Spatial structure, cooperation and competition in biofilms. Nat Rev Microbiol 14, 589-600 (2016).
6 Waters, C. M. & Bassler, B. L. Quorum sensing: cell-to-cell communication in bacteria. Annu Rev Cell Dev Biol 21, 319-346 (2005).
7 Papenfort, K. & Bassler, B. L. Quorum sensing signal-response systems in Gram-negative bacteria. Nat Rev Microbiol 14, 576-588 (2016).
8 Novick, R. P. & Geisinger, E. Quorum sensing in Staphylococci. Annu Rev Genet 42, 541-564 (2008).
10 Arvidson, S. & Tegmark, K. Regulation of virulence determinants in Staphylococcus aureus. Int J Med Microbiol 291, 159-170 (2001).
11 Hammer, B. K. & Bassler, B. L. Quorum sensing controls biofilm formation in Vibrio cholerae. Mol Microbiol 50, 101-104 (2003).
12 Hsiao, A. et al. Members of the human gut microbiota involved in recovery from Vibrio cholerae infection. Nature 515, 423-426 (2014).
13 Ismail, A. S., Valastyan, J. S. & Bassler, B. L. A host-produced autoinducer-2 mimic activates bacterial quorum sensing. Cell Host Microbe 19, 470-480 (2016).
14 O'Loughlin, C. T. et al. A quorum-sensing inhibitor blocks Pseudomonas aeruginosa virulence and biofilm formation. Proc Natl Acad Sci USA 110, 17981-17986 (2013).
15 Sully, E. K. et al. Selective chemical inhibition of agr quorum sensing in Staphylococcus aureus promotes host defense with minimal impact on resistance. Plos Pathog 10 (2014).
16 Otto, M. Quorum-sensing control in Staphylococci—a target for antimicrobial drug therapy? Fems Microbiol Lett 241, 135-141 (2004).
17 Cegelski, L., Marshall, G. R., Eldridge, G. R. & Hultgren, S. J. The biology and future prospects of antivirulence therapies. Nature Reviews Microbiology 6, 17-27 (2008).
18 Kim, M. K., Ingremeau, F. B., Zhao, A., Bassler, B. L. & Stone, H. A. Local and global consequence of flow on bacterial quorum sensing. Nature Microbiology 1, 15005 (2016).
19 Lu, H. D. et al. Modulating Vibrio cholerae quorum-sensing-controlled communication using autoinducer-loaded nanoparticles. Nano Lett 15, 2235-2241 (2015).
20 Persat, A. et al. The mechanical world of bacteria. Cell 161, 988-997 (2015).
21 Kim, M. K., Drescher, K., Pak, O. S., Bassler, B. L. & Stone, H. A. Filaments in curved streamlines: rapid formation of Staphylococcus aureus biofilm streamers. New J Phys 16 (2014).
22 Broderick, A. H. et al. Surface coatings that promote rapid release of peptide-based AgrC inhibitors for attenuation of quorum sensing in Staphylococcus aureus. Adv Healthc Mater 3, 97-105 (2014).
23 Ho, K. K. K. et al. Quorum sensing inhibitory activities of surface immobilized antibacterial dihydropyrrolones via click chemistry. Biomaterials 35, 2336-2345 (2014).
24 Lowy, F. D. Staphylococcus aureus infections. New Engl J Med 339, 2026-2027 (1998).
25 Gordon, R. J. & Lowy, F. D. Pathogenesis of methicillin-resistant Staphylococcus aureus infection. Clin Infect Dis 46, S350-S359 (2008).
26 Zhang, L. S., Gray, L., Novick, R. P. & Ji, G. Y. Transmembrane topology of AgrB, the protein involved in the post-translational modification of AgrD in Staphylococcus aureus. J Biol Chem 277, 34736-34742 (2002).
27 Wang, B. Y., Zhao, A. S., Novick, R. P. & Muir, T. W. Key driving forces in the biosynthesis of autoinducing peptides required for staphylococcal virulence. P Natl Acad Sci USA 112, 10679-10684 (2015).
28 Lina, G. et al. Transmembrane topology and histidine protein kinase activity of AgrC, the agr signal receptor in Staphylococcus aureus. Mol Microbiol 28, 655-662 (1998).
29 Wang, B. Y., Zhao, A. S., Novick, R. P. & Muir, T. W. Activation and inhibition of the receptor histidine kinase AgrC occurs through opposite helical transduction motions. Mol Cell 53, 929-940 (2014).
30 Koenig, R. L., Ray, J. L., Maleki, S. J., Smeltzer, M. S. & Hurlburt, B. K. Staphylococcus aureus AgrA binding to the RNAIII-agr regulatory region. J Bacteriol 186, 7549-7555 (2004).
31 Fechter, P., Caldelari, I., Lioliou, E. & Romby, P. Novel aspects of RNA regulation in Staphylococcus aureus. Febs Lett 588, 2523-2529 (2014).
32 Lyon, G. J., Wright, J. S., Muir, T. W. & Novick, R. P. Key determinants of receptor activation in the agr autoinducing peptides of Staphylococcus aureus. Biochemistry-Us 41, 10095-10104 (2002).
33 Hong, V., Presolski, S. I., Ma, C. & Finn, M. G. Analysis and optimization of copper-catalyzed azide-alkyne cycloaddition for bioconjugation. Angew Chem Int Edit 48, 9879-9883 (2009).
34 Emilsson, G. et al. Strongly stretched protein resistant poly(ethylene glycol) brushes prepared by grafting-to. Acs Appl Mater Inter 7, 7505-7515 (2015).
35 Cisar, E. A., Geisinger, E., Muir, T. W. & Novick, R. P. Symmetric signalling within asymmetric dimers of the Staphylococcus aureus receptor histidine kinase AgrC. Mol Microbiol 74, 44-57 (2009).
36 Vollmer, W., Blanot, D. & de Pedro, M. A. Peptidoglycan structure and architecture. Fems Microbiol Rev 32, 149-167 (2008).
37 Traber, K. E. et al. agr function in clinical Staphylococcus aureus isolates. Microbiol-Sgm 154, 2265-2274 (2008).
38 Khodaparast, S., Kim, M. K., Silpe, J. & Stone, H. A. Bubble-driven detachment of bacteria from confined micro-geometries. Submitted (2016).
39 Vasilev, K., Cook, J. & Griesser, H. J. Antibacterial surfaces for biomedical devices. Expert Rev Med Devic 6, 553-567 (2009).
40 Gallo, J., Holinka, M. & Moucha, C. S. Antibacterial surface treatment for orthopaedic implants. Int J Mol Sci 15, 13849-13880 (2014).
41 Lee, J. J. et al. Synthetic ligand-coated magnetic nanoparticles for microfluidic bacterial separation from blood. Nano Lett 14, 1-5 (2014).
42 Boles, B. R. & Horswill, A. R. agr-mediated dispersal of Staphylococcus aureus biofilms. Plos Pathog 4 (2008).
43 Davies, D. Understanding biofilm resistance to antibacterial agents. Nat Rev Drug Discov 2, 114-122 (2003).
44 Huycke, M. M., Spiegel, C. A. & Gilmore, M. S. Bacteremia caused by hemolytic, high-level gentamicin-resistant Enterococcus faecalis. Antimicrob Agents Ch 35, 1626-1634 (1991).
45 Cook, L. C. & Federle, M. J. Peptide pheromone signaling in Streptococcus and Enterococcus. Fems Microbiol Rev 38, 473-492 (2014).
46 Mylonakis, E. et al. The Enterococcus faecalis fsrB gene, a key component of the fsr quorum-sensing system, is associated with virulence in the rabbit endophthalmitis model. Infect Immun 70, 4678-4681 (2002).
47 Nakayama, J. et al. Development of a peptide antagonist against fsr quorum sensing of Enterococcus faecalis. ACS Chem Biol 8, 804-811 (2013).
48 Gray, B., Hall, P. & Gresham, H. Targeting agr- and agr-like quorum sensing systems for development of common therapeutics to treat multiple Gram-positive bacterial infections. Sensors-Basel 13, 5130-5166 (2013).
49 Fujii, T. et al. Two homologous agr-like quorum-sensing systems cooperatively control adherence, cell morphology, and cell viability properties in Lactobacillus plantarum WCFS1. J Bacteriol 190, 7655-7665 (2008).
50 Wang, I. N., Smith, D. L. & Young, R. Holins: The protein clocks of bacteriophage infections. Annu Rev Microbiol 54, 799-825 (2000).
51 Goedhart, J. et al. Structure-guided evolution of cyan fluorescent proteins towards a quantum yield of 93%. Nature Communications 3 (2012).
52 Nadell, C. D., Drescher, K., Wingreen, N. S. & Bassler, B. L. Extracellular matrix structure governs invasion resistance in bacterial biofilms. Isme Journal 9, 1700-1709 (2015).
53 Chen, J., Yoong, P., Ram, G., Tones, V. J. & Novick, R. P. Single-copy vectors for integration at the SaPI1 attachment site for Staphylococcus aureus. Plasmid 76, 1-7 (2014).
54 Lyon, G. J., Mayville, P., Muir, T. W. & Novick, R. P. Rational design of a global inhibitor of the virulence response in Staphylococcus aureus, based in part on localization of the site of inhibition to the receptor-histidine kinase, AgrC. P Natl Acad Sci USA 97, 13330-13335 (2000).
S1 Kreiswirth, B. N. et al. The toxic shock syndrome exotoxin structural gene is not detectably transmitted by a prophage. Nature 305, 709-712 (1983).
S2 Ruzin, A., Lindsay, J. & Novick, R. P. Molecular genetics of SaPl1—a mobile pathogenicity island in Staphylococcus aureus. Mol Microbiol 41, 365-377 (2001).
S3 Novick, R. P. Genetic Systems in Staphylococci. Method Enzymol 204, 587-636 (1991).
S4 Novick, R. P. et al. Synthesis of staphylococcal virulence factors is controlled by a regulatory RNA molecule. Embo J 12, 3967-3975 (1993).
S5 Kim, M. K., Ingremeau, F. B., Zhao, A., Bassler, B. L. & Stone, H. A. Local and global consequences of flow on bacterial quorum sensing. Nature Microbiology 1, 15005 (2016).
S6 Lyon, G. J., Mayville, P., Muir, T. W. & Novick, R. P. Rational design of a global inhibitor of the virulence response in Staphylococcus aureus, based in part on localization of the site of inhibition to the receptor-histidine kinase, AgrC. P Natl Acad Sci USA 97, 13330-13335 (2000).
S7 Geisinger, E., George, E. A., Muir, T. W. & Novick, R. P. Identification of ligand specificity determinants in AgrC, the Staphylococcus aureus quorum-sensing receptor. J Biol Chem 283, 8930-8938 (2008).

What is claimed is:

1. A QS modulating conjugate comprising a QS modulating molecule attached to a trackable moiety, wherein the QS modulating conjugate binds to a receptor on a surface of a bacterial cell.

2. (canceled)

3. The bacterial cell of claim 1, wherein the bacterial cell is Gram-positive or Gram-negative.

4. The bacterial cell of claim 3, wherein the Gram-positive bacterial cell is exposed to a permeability agent that forms holes in the outer membrane layer of the bacterial cell.

5. The QS modulating conjugate of claim 1, further comprising a linker that joins the QS modulating molecule to the trackable moiety.

6. The QS modulating conjugate of claim 5, wherein the linker has (a) a diameter of less than 5 nm and/or (b) a length greater than 15 nm.

7. (canceled)

8. The QS modulating conjugate of claim 5, wherein the linker is selected from polyethylene glycol (PEGs), polyphosphazenes, polylactide, polyglycolide, polycaprolactone, or any other combinations thereof.

9. The QS modulating conjugate of claim 1, wherein the QS modulating conjugate comprises an antagonist or an agonist of QS, biofilm production, biofilm streamer production and/or virulence factor production.

10. (canceled)

11. The QS modulating conjugate of claim 1, comprising a second QS modulating conjugate that (a) competitively binds to the receptor and/or (b) binds to a different receptor on a different bacterial cell.

12. (canceled)

13. The QS modulating conjugate of claim 1, wherein the QS modulating molecule exhibits modulating activity on the bacteria cell.

14. The QS modulating conjugate of claim 1, wherein the QS modulating conjugate is formed by attaching a molecule selected from Tables 1A or 1B or its derivative molecules to the trackable moiety.

15. The QS modulating conjugate of claim 1, wherein the QS modulating molecule is attached to the trackable moeity using one or more of the following types of chemical reactions: biorthogonal reactions, click chemistry, thiol-ene reactions, gold-sulfide bond formation, esterification reactions, Grignard reactions, Michael reactions, ketone/hydroxylamine condensations, Staudinger ligations, strain-promoted alkyne-azide cycloadditions, photo-click cycloadditions, Diels-Alder cycloadditions, tetrazine-alkene/alkyne cycloadditions, Cu-catalyzed alkyne-azide cycloadditions, Pd-catalyzed cross coupling, strain promoted alkyne-nitrone cycloadditions, Cross-metathesis, Norbornene cycloadditions, Oxanorbornadiene cycloadditions, tetrazine ligations, or tetrazole photoclick chemistry.

16. The QS modulating conjugate of claim 1, wherein the trackable moiety is a fluorophore, a radionuclide or a PET probe and/or (b) wherein the QS modulating molecule is covalently attached to the trackable moiety.

17. (canceled)

18. The QS modulating conjugate of claim 1, wherein the QS modulating conjugate is placed in an environment that is an implantable medical device, part of machinery used in industrial processes, a culvert, a pool used in a waste water treatment facility, waste water treatment facility, a pipe, a cooling tower, a medical device, industrial fluid handling machinery, a wound, within the body, a medical process, an agricultural processes, and/or machinery.

19. Use of the QS modulating conjugate of claim 1 to (a) promote or inhibit pathogenic behaviors of a microorganism (b) promote beneficial behaviors of a microorganism; and/or (c) promote or inhibit biofilm formation.

20. (canceled)

21. The use of the QS modulating conjugate of claim 19, wherein the microorganism is selected from bacteria, archaea, protozoa, fungi, and/or algae.

22. The use of the QS modulating conjugate of claim 21, wherein the bacteria is selected from Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anabaena, Anabaenopsis, Anaerobospirillum, Anaerorhabdus, Aphanizomenon, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Bordetella, Borrelia, Brachyspira, Branhamella, Brevibacillus, Brevibacterium, Brevundimonas, Brucella, Burkholderia, Buttiauxella, Butyrivibrio, Calymmatobacterium, Camesiphon, Campylobacter, Capnocytophaga, Capnylophaga, Cardiobacterium, Catonella, Cedecea, Cellulomonas, Centipeda, Chlamydia, Chlamydophila, Chromobacterium, Chryseomonas, Chyseobacterium, Citrobacter, Clostridium, Collinsella, Comamonas, Corynebacterium, Coxiella, Cryptobacterium, Cyanobacteria, Cylindrospermopsis, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gloeobacter, Gordona, Haemophilus, Hafnia, Hapalosiphon, Helicobacter, Helococcus, Hemophilus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptospirae, Leptotrichia, Leuconostoc, Listeria, Listonella, Lyngbya, Megasphaera, Methylobacterium, Microbacterium, Micrococcus, Microcystis, Mitsuokella, Mobiluncus, Moellerella, Moraxella, Morganella, Mycobacterium, Mycoplasma, Myroides, Neisseria, Nocardia, Nocardiopsis, Nodularia, Nostoc, Ochrobactrum, Oeskovia, Oligella, Orientia, Paenibacillus, Pantoea, Parachlamydia, Pasteurella, Pediococcus, Peptococcus, Peptostreptococcus, Phormidium, Photobacterium, Photorhabdus, Phyllobacterium, Phytoplasma, Planktothrix, Plesiomonas, Porphyromonas, Prevotella, Propionibacterium, Proteus, Providencia, Pseudoanabaena, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Schizothrix, Selenomonas, Serpulina, Serratia, Shewenella, Shigella, Simkania, Slackia, Sphaerotilus, Sphingobacterium, Sphingomonas, Spirillum, Spiroplasma, Spirulina, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Trichodesmium, Tropheryma, Tsakamurella, Turicella, Umezakia, Ureaplasma, Vagococcus, Veillonella, Vibrio, Weeksella, Wolinella, Xanthomonas, Xenorhabdus, Yersinia, Yokenella. Acinetobacter baumannii, Actinobacillus actinomycetemcomitans, Actinobacillus pleuropneumoniae, Actinomyces bovis, Actinomyces israelii, Bacillus anthracis, Bacillus ceretus, Bacillus coagulans, Bacillus liquefaciens, Bacillus popillae, Bacillus subtilis, Bacillus thuringiensis, Bacteroides distasonis, Bacteroides fragilis, Bacteroides thetaiotaomicron, Bacteroides vulgatus, Bartonella bacilliformis, Bartonella Quintana, Beneckea parahaemolytica, Bordetella bronchiseptica, Bordetella parapertussis, Bordetella pertussis, Borelia burgdorferi, Brevibacterium lactofermentum, Brucella abortus, Brucella canis, Brucella melitensis, Brucella suis, Burkholderia cepacia, Burkholderia mallei, Burkholderia pseudomallei, Campylobacter fetus, Campylobacter jejuni, Campylobacter pylori, Cardiobacterium hominis, Chlamydia pneumoniae, Chlamydia psittaci, Chlamydia trachomatis, Chlamydophila abortus, Chlamydophila caviae, Chlamydophila felis, Chlamydophila pneumonia, Chlamydophila psittaci, Chryseobacterium eningosepticum, Clostridium botulinum, Clostridium butyricum, Clostridium coccoides, Clostridium difficile, Clostridium leptum, Clostridium tetani, Corynebacterium xerosis, Cowdria ruminantium, Coxiella burnetii, Edwardsiella tarda, Ehrlichia sennetsu, Eikenella corrodens, Elizabethkingia meningoseptica, Enterobacter aerogenes, Enterobacter cloacae, Enterococcus faecalis, Escherichia coli, Escherichia hirae, Flavobacterium meningosepticum, Fluoribacter bozemanae, Francisella tularensis, Francisella tularensis biovar Tularensis, Francisella tularensis subsp. Holarctica, Francisella tularensis subsp. nearctica, Francisella tularensis subsp. Tularensis, Francisella tularensis var. palaearctica, Fudobascterium nucleatum, Fusobacterium necrophorum, Haemophilus ducreyi, Haemophilus influenzae, Helicobacter pylori, Kingella kingae, Klebsiella mobilis, Klebsiella oxytoca, Klebsiella pneumoniae, Lactobacillus acidophilus, Lactobacillus casei, Lactobacillus hilgardii, Lactobacillus pentosus, Lactobacillus plantarum, Lactobacillus rhamnosus, Lactococcus lactis, Legionella bozemanae corrig., Legionella pneumophila, Leptospira alexanderi, Leptospira borgpetersenii, Leptospira fainei, Leptospira inadai, Leptospira interrogans, Leptospira kirschneri, Leptospira noguchii, Leptospira santarosai, Leptospira weilii, Leuconostoc lactis, Leuconostoc oenos, Listeria ivanovii, Listeria monocytogenes, Moraxella catarrhalis, Morganella morganii, Mycobacterium africanum, Mycobacterium avium, Mycobacterium avium subspecies paratuberculosis, Mycobacterium bovis, Mycobacterium bovis strain BCG, Mycobacterium intracellulare, Mycobacterium kansasii, Mycobacterium leprae, Mycobacterium marinum, Mycobacterium tuberculosis, Mycobacterium typhimurium, Mycobacterium ulcerans, Mycoplasma hominis, Mycoplasma mycoides, Mycoplasma pneumoniae, Neisseria gonorrhoeae, Neisseria meningitidis, Neorickettsia sennetsu, Nocardia asteroides, Orientia tsutsugamushi, Pasteurella haemolytica, Pasteurella multocida, Plesiomonas shigelloides, Propionibacterium acnes, Proteus mirabilis, Proteus morganii, Proteus penneri, Proteus rettgeri, Proteus vulgaris, Providencia alcalifaciens, Providencia rettgeri, Pseudomonas aeruginosa, Pseudomonas mallei, Pseudomonas pseudomallei, Pyrococcus abyssi, Rickettsia akari, Rickettsia canadensis, Rickettsia canadensis corrig, Rickettsia conorii, Rickettsia montanensis, Rickettsia montanensis corrig, Rickettsia prowazekii, Rickettsia rickettsii, Rickettsia sennetsu, Rickettsia tsutsugamushi, Rickettsia typhi, Rochalimaea quintana, Salmonella arizonae, Salmonella choleraesuis subsp. arizonae, Salmonella enterica subsp. Arizonae, Salmonella enteritidis, Salmonella paratyphi, Salmonella typhi, Salmonella typhimurium, Selenomonas nominantium, Selenomonas ruminatium, Serratia marcescens, Shigella boydii, Shigella dysenteriae, Shigella flexneri, Shigella sonnei, Spirillum minus, Staphylococcus aureus, Staphylococcus epidermidis, Staphylococcus equi, Staphylococcus lugdunensis, Stenotrophomonas maltophila, Streptobacillus moniliformis, Streptococcus agalactiae, Streptococcus bovis, Streptococcus ferus, Streptococcus pneumoniae, Streptococcus pyogenes, Streptococcus viridans, Streptomyces ghanaenis, Streptomyces hygroscopicus, Streptomyces phaechromogenes, Treponema carateum, Treponema denticola, Treponema pallidum, Treponema pertenue, Vibrio cholerae, Vibrio parahaemolyticus, Vibrio vulnificus, Xanthomonas maltophilia, Yersinia enterocolitica, Yersinia pestis, Yersinia pseudotuberculosis, Zymomonas mobilis, or Fusospirochetes.

23. The use of the QS modulating conjugate of claim 21, wherein the fungi is selected from Candida, Saccharomyces, or Cryptococcus.

24. The use of the QS modulating conjugate of claim 19, wherein the conjugate is used to treat sepsis, pneumonia, infections from cystic fibrosis, otitis media, chronic obstructive pulmonary disease, a urinary tract infection, periodontal disease, gingivitis, periodontitis, breath malodor, treat infections, Gram-negative infections, Gram-positive infections, otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), cystic fibrosis, an accumulation of biofilm in the lungs or digestive tract, emphysema, chronic bronchitis, also encompasses infections on implanted/inserted devices, medical device-related infections, biliary stent infections, orthopedic implant infections, catheter-related infections, skin infections, dermatitis, ulcers from peripheral vascular disease, a burn injury, trauma, rosacea, skin infection, pneumonia, otitis media, sinusitus, bronchitis, tonsillitis, and mastoiditis related to infection by Streptococcus pneumoniae, Haemophilus influenzae, Moraxella catarrhalis, Staphylococcus aureus, Peptostreptococcus spp. or Pseudomonas spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), S. pyogenes, S. agalactiae, Streptococcal groups C-F (minute-colony streptococci), viridans streptococci, Corynebacterium spp., Clostridium spp., or Bartonella henselae; uncomplicated acute urinary tract infections related to infection by S. saprophyticus or Enterococcus spp.; urethritis and cervicitis; sexually transmitted diseases related to infection by Chlamydia trachomatis, Haemophilus ducreyi, Treponema pallidum, Ureaplasma urealyticum, or Nesseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by C. trachomatis, N. gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringens or Bacteroides spp.; skin infection by S. aureus, Propionibacterium acne; atherosclerosis related to infection by Helicobacter pylori or Chlamydia pneumoniae; or the like.

25. The use of the QS modulating conjugate of claim 1 for detecting the presence of a specific microorganism in a patient.

26. The use of the QS modulating conjugate of claim 18, wherein the detecting comprises (a) detecting a QS modulating conjugate comprising a radionuclide bound to the surface of the microorganism and/or (b) detecting a QS modulating conjugate comprising a fluorophore bound to the surface of the microorganism.

27. (canceled)

28. The use of the QS modulating conjugate of claim 1 for screening for a test compound that modulates QS, biofilm formation, biofilm streamer formation, and/or a virulence factor production by a microorganism.


SEQUENCE LISTING (12 sequences)

SEQ ID NO: 1 - DNA, 59 bp, Artificial Sequence
  Primer
tcgttaactaattaatttaagaaggagatatacatatggtatcaaaaggggaagagttg

SEQ ID NO: 2 - DNA, 40 bp, Artificial Sequence
  Primer
ttagaataggcgcgccttatttgtacagttcgtccatgcc

SEQ ID NO: 3 - DNA, 62 bp, Artificial Sequence
  Primer
tcgttaactaattaatttaagaaggagatatacatatgagtaaaggagaagaacttttcact

SEQ ID NO: 4 - DNA, 46 bp, Artificial Sequence
  Primer
ttagaataggcgcgccttattatttgtatagttcatccatgccatg

SEQ ID NO: 5 - DNA, 59 bp, Artificial Sequence
  Primer
tcgttaactaattaatttaagaaggagatatacatatggtgagtgtgattaaaccagag

SEQ ID NO: 6 - DNA, 42 bp, Artificial Sequence
  Primer
ttagaataggcgcgccttaggaatgagctactgcatcttcta

SEQ ID NO: 7 - DNA, 39 bp, Artificial Sequence
  Primer
tataatagcatgcacataacaccaaaaagaagaaggtgc

SEQ ID NO: 8 - DNA, 36 bp, Artificial Sequence
  Primer
ccgcaaaggcgcctgtcactttgcttgatatatgag

SEQ ID NO: 9 - DNA, 60 bp, Artificial Sequence
  Primer
aatacgccgttaactgactttattatcttattatatttttttaacgtttctcaccgatgc

SEQ ID NO: 10 - DNA, 60 bp, Artificial Sequence
  Primer
ggaggggctcacgaccatacttacatgtcaacgataatacaaaatataatacaaaatata

SEQ ID NO: 11 - DNA, 60 bp, Artificial Sequence
  Primer
ttgaatacgccgttaactgactttattatcttattatatttttttaacgtttctcaccga

SEQ ID NO: 12 - DNA, 23 bp, Artificial Sequence
  Primer
ggaggggctcacgaccatactta